(19) United States
(12) Patent Application Publication
(10) Pub. No.: US 2025/0058010 A1
(43) Pub. Date: Feb. 20, 2025

(54) SYSTEM AND METHOD FOR ABATEMENT OF ALLERGENS, PATHOGENS AND VOLATILE ORGANIC COMPOUNDS

(71) Applicant: Prompt Care, Inc., Cayucos, CA (US)

(72) Inventors: David W. BRANDMEYER, Cayucos, CA (US); Benedict B. ST. ONGE, Cayucos, CA (US); Richard P. HOOPER, Cayucos, CA (US); Daniel N. BRANDMEYER, Cayucos, CA (US)

(21) Appl. No.: 18/720,532

(22) PCT Filed: Dec. 15, 2022
(86) PCT No.: PCT/US2022/053086

§ 371 Date: Jun. 14, 2024

Related U.S. Application Data
(60) Provisional application No. 63/290,917, filed on Dec. 17, 2021.

(51) Int. Cl. A61L 2/24; A61L 2/20; A61L 9/015; A61L 9/14

(52) U.S. Cl. CPC A61L 2/24; A61L 2/202; A61L 2/208; A61L 9/015; A61L 9/14; A61L 2202/11; A61L 2202/14; A61L 2202/15; A61L 2202/25; A61L 2209/111; A61L 2209/16; A61L 2209/211; A61L 2209/212

(57) ABSTRACT

An air and surface treatment system including a Peroxide Humidifier and an Ozone Generator is disclosed herein. The Peroxide Humidifier and Ozone Generator may be configured to communicate with one another. The Peroxide Humidifier and Ozone Generator may be configured to communicate with a Worksite Control Device and an external server. The Peroxide Humidifier, Ozone Generator, Equipment Control Device, and/or the Worksite Control Device may be configured to communicate with other devices. The concepts introduced by this application greatly enhance the safety and operational efficacy of the equipment by utilizing computing resources to control the application process. Methods for using the devices and systems are also disclosed.

### BACKGROUND

### Field

The field pertains to air and exposed surface treatment. More particularly, the field pertains to a system for treating air and all exposed surfaces with hydrogen peroxide and/or ozone in combination to form hydroxyl radicals for the reduction of chemical bonds under control of an advanced computer system.

### Background

Systems for air and surface treatment, for example, to remove allergens, pathogens, odors, dust, and other such unwanted contaminants are particularly advantageous for buildings which have been damaged by mold or smoke or have been exposed to other airborne contaminants, such as those from cooking, cleaning, and home or industrial use of chemicals. However, these conventional systems exhibit certain drawbacks, including but not limited to safety, efficiency, and cost. Conventional methods include the use of chlorine bleach on all contained surfaces. This is labor intensive and only reaches the exterior of smooth surfaces walls and objects and is not advised for absorbent or complex surfaces. Another method involves the use of chlorine dioxide gas as a fumigant in enclosed areas. Chlorine dioxide is less effective than ozone and can leave an objectionable odor after treatment. It is also known to leave a yellow stain on some plastics and cloth materials. Additionally, it requires longer treatment cycles as compared to ozone and hydrogen peroxide generated hydroxyl radical treatments.

### SUMMARY

The methods and apparatus provided enhance the operation and safety of the equipment, e.g., by utilizing computing resources to control the application process. Data acquired from the array of sensors embedded in the Peroxide Humidifiers and Ozone Generators in conjunction with one or more Equipment Control Device(s) and one or more Worksite Control Device(s) to log data sets to a central computer server can be employed. Equipment sensors located in the Peroxide Humidifiers and Ozone Generators utilize the communication and signal coordination system provided by the Equipment Control Device to process sensor signals information. These three subsystems operate as a set in the treatment process obtaining data prior to communications with the Worksite Control Device before passing onto the central computer or computer systems connected by wireless and wired communications protocols.

To treat the air and all exposed surfaces, portable treatment equipment is typically brought to the building and left to operate for some time. Those operating such systems may not stay with the equipment for the entire duration of the treatment because of the breathing hazard present by the oxidizing agents. In another embodiment the equipment is fixed in place incorporated into new or existing building systems, potentially working in conjunction with existing equipment, except during the treatment process where it takes command of the enclosed areas servicing one or more rooms, including up to the entire building structure.

The air treatment system introduces oxidizing agents into the air, such as ozone and hydrogen peroxide. In combination, ozone and hydrogen peroxide form hydroxyl radicals that form at any point in the interior enclosed space including exposed surfaces where they can break down complex chemical bonds on contact. Other flawed systems generate ozone alone with ultra-violet (UV) light. Exposed UV light can be harmful to people, animals, and plants. UV light is only efficient at treating line-of-site object surfaces leaving shaded surfaces untouched. UV lamps are also capable of generating ozone gas from room air. Other systems dispense only hydrogen peroxide, and while the agents are helpful for treating the air, if spilled in high concentration, the oxidizing agents can cause damage to various objects, such as furniture and floors. Only under proper combinations can hydroxyl radicals form and treat the air and exposed surfaces, (e.g., flat surfaces, such as walls, countertops, floors, ceilings, as well as surfaces of pores, holes, fibers, surfaces of tortuous spaces, or anywhere else that a gas can penetrate), rendering them free from offending compounds or microorganisms under an efficient repeatable process. Spot treatment of specific areas involving other oxidizers like chlorine bleach or pet urine neutralizing products may also be used on offending substances in high concentration areas on specific surfaces prior to the treatment process.

The safety of both the operator and surrounding community is highly important. The ability to initiate treatment cycles from outside the enclosed space via the integrated computing system, allows the operator to remain outside the structure initiating operations from a portable operator interface like a tablet device running treatment code. This prevents direct operator exposure to the oxidizers present during treatment.

In the fixed place embodiment, the operator enters commands into a Technician Interface Device, usually physically mounted outside the treatment area, and the operation is initiated either locally by the Technician Interface Device or by a local or remote server, communicating start/stop operations as well as room access and locking functions. Like the portable version equipment, the same sensors and control functions are available in the fixed in place unit except the equipment is integrated into the building itself and not moved between treatments. This implementation relies on biometrics or other appropriately secure access controls to limit access to the treatment area during treatments. The operator is not exposed at any time to the treatment process because of the communication, local sensors, and automation processes. The Technician Interface Device can alternatively be a portable computer or handheld device (e.g., a smart phone), or any other suitable computing device. The computing device may optionally be equipped with a device to determine proximity to or position relative to the treatment area, e.g., GPS, NFC, or Bluetooth. Equipment readiness and preventative maintenance is monitored by computer systems and sensor technology incorporated within the treatment process.

Automated data logging reduces the operator interaction required. Eliminating operator entries further reduces the human errors associated with manual entries.

Smart artificial intelligent systems continuously monitor process data to alert an operator of any anomalies that could have safety or efficacy impacts on the process, equipment, or the enclosed space being treated. The expert artificial intelligence system located in the server, or elsewhere but in communication with the server or other computing device, e.g., in the cloud, monitors the condition of the overall treatment process and equipment status and can determine a large range of anomalies occurring in conjunction with the hardware operation.

Multiple site treatments can now be done simultaneously as equipment is under control of an expert system remotely controlled by an operator interface that is able to communicate with the device or devices directly or through any other means, including but not limited to wired or wireless internet connectivity, or cellular data connection. Having a number of Peroxide Humidifiers, Ozone Generators, and Equipment Control Devices subsystems in conjunction with one or more Worksite Control Device at each location, facilitates one operator to monitor and control a number of treatment operations at one time. These need not be located at the same site but in a close geographic area for efficient use of time and resources. One operator could efficiently operate multiple treatment cycles at one time in a mobile operation. In the fixed building installation, the number of simultaneous treatments is not limited by a single operator, so the number of operations is limited only by internet connectivity channels available. In certain embodiments, a centrally located operator can control treatments at multiple treatment locations, e.g., geographically dispersed locations for one or more clients.

Equipment intelligence now prevents damaged equipment from being used in a treatment process by analyzing data from a previous or an in-process treatment to alert the operator of a problem, malfunction, anomalous situation, or need for maintenance (either scheduled or in response to data collected). Malfunctioning equipment can be electronically tagged for repair or replacement by detecting anomalies in its operation and prevented from inadvertently being used for a later treatment process.

Equipment intelligence prevents an operator from accidentally leaving equipment behind during the treatment completion packing process. Similar intelligence can be employed to prevent equipment from being deployed in an incorrect location, or to assist in recovering stolen equipment.

Automated systems analyze the data gathered by the treatment devices using methods that include artificial intelligence to predict and anticipate the necessary maintenance of the treatment devices, allowing more efficient treatment. A self-test operation can be initiated before, during or after a treatment cycle to ensure the hardware is working within performance parameters for each device involved in the treatment process. Any anomalies will be assessed to determine the impact on the current and future treatment for efficacy, safety, and severity of the anomaly. Maintenance priority assessments are then processed and scheduled accordingly.

The smart system described here also provides for dynamic rescheduling, billing, and tracking ongoing recurring treatment processes for each client. Because diverse types of treatments require distinct treatment profiles, the system can recommend a profile for new clients based on previous actions taken on similar treatment projects. This could involve local climate, elevation, special weather circumstances. This smart system along with the test operator feedback will lead to system optimization improvements over time not possible with the hardware system alone.

The smart software systems employed in this application control each piece of equipment individually to allow tailoring of an applications processes tailored for an establishment (company SOP “Standard Operating Procedures”) to fit into existing and specific requirements dictated by existing processes and procedures associated with a group of clients (example—an existing company directive followed by all employees of that establishment) This added software greatly simplifies the process of adding new clients with existing standard operating procedure (SOP) to provide treatment services.

The smart equipment processes have been designed to treat both fixed installations and mobile applications using mobile transit case-based equipment and dedicated fixed in place equipment for regular periodic treatments. Control code and human interfaces are adapted to best accommodate the specific requirements of each. Features of the fixed equipment systems can be employed in mobile equipment systems, and vice versa.

Sensors implemented in ozone and hydrogen peroxide subsystems include, but are not limited to, temperature, tank depth, current sensing, photo image, proximity, audio, motion, and other measurable aspects of the treatment area to monitor the status of the equipment.

Within the ozone generation subsystem, a current monitoring system keeps the status of the lamps always known. As the lamps age, the power consumption and UV output drops linearly with age. Some of this aging effect can be mitigated by adjusting the current to the lamps to maintain optical power. When a lamp fails, an abrupt change in the current from the last operation is observed. A temperature sensor above the lamps monitors the inside temperature in the event the device is tipped over or the internal fan fails to rotate properly. A solid-state relay or mechanical contactor under computer control activates the device when commanded by a wireless communication protocol. Attempts at correcting temperature problems can be made by the software system to recover operation by controlling the lamp current or briefly interrupting the power to the lamps in an attempt to rectify internal temperature anomalies.

Within the peroxide dispenser sub system, the level of the peroxide is monitored via an E-taps resistive depth sensor. Other means of depth sensing using ultrasonic, time of flight optical, pressure sensors or floats could be used as well.

Temperature sensors like the chips type TMP 36 are placed on the inlet and outlet to monitor the evaporation rate. The humidity in the room is measured by knowing the delta temperature between the 2 sensors giving an equivalent to wet bulb and dry bulb information data. Other temperature sensors, including but limited to alcohol direct read thermometers, digital conversion circuit sensors using diodes as the sensor or other means of temperature monitoring can also be used.

Failure in a pump or fan mechanism can be detected by the lack of a delta temperature after the system reaches stability operation of 5 minutes. A solid-state relay, mechanical contactor, or pulse width modulation device is incorporated to activate the fan and pump devices remotely by a wireless communication protocol.

Additional sensors, including but not limited to cameras and motion sensors, help to increase the safety of the treatment process by ensuring the premise stays evacuated during the treatment process.

### BRIEF DESCRIPTION OF THE DRAWINGS

FIG. 1 is a block diagram of a system used for treating air.

FIG. 2 is a schematic diagram illustrating one embodiment of an Ozone Generator for use in the system of FIG. 1.

FIGS. 3A and 3B are schematic diagrams, each respectively illustrating embodiments of Peroxide Humidifiers for use in the system of FIG. 1.

FIG. 3C is a schematic diagram of an embodiment of a diffuser for use in the Peroxide Humidifier of FIG. 3B.

FIG. 4 is a schematic diagram of a system used for treating air and surfaces.

FIG. 5 is a diagram of features of one embodiment of an Ozone Generator.

FIG. 6A depicts a Pre-treatment Process Flow.

FIG. 6B depicts a Treatment Process Flow prior to start.

FIG. 6C depicts a Treatment Process Flow during treatment.

FIG. 6D depicts a Treatment Process Flow at the end of treatment.

FIG. 6E depicts an overall Treatment Process Flow.

FIG. 7 is a diagram of features of one embodiment of an Ozone Generator.

FIG. 8 is a rendition of a system incorporated into a building infrastructure.

FIG. 9 depicts an Ozone Generator case.

FIG. 10 depicts a Peroxide Humidifier case.

FIGS. 11A and 11B depict one embodiment of a Worksite Control Device.

FIG. 12 is a schematic illustration of inventive aspects of a lamp which can be used for the Ozone Generator of FIG. 2.

FIG. 13 is a schematic illustration of a dual tank peroxide humidifier.

### DETAILED DESCRIPTION

In one aspect an air treatment system includes a Peroxide Humidifier and an Ozone Generator pair. Both devices need to operate together in a minimum combination of one of each or in a single device with both functions collocated. The Peroxide Humidifier includes communication circuitry integrated therewith, and the Ozone Generator includes communication circuitry integrated therewith. This circuitry is in the form of a microcontroller (Raspberry Pi, Arduino, or other similar device) with internal hardware bus communication devices under software control. In addition, the Peroxide Humidifier and the Ozone Generator are configured to communicate with each other via a Worksite Control Device. This device incorporates a microcontroller with communications hardware under software control.

In some embodiments, the Peroxide Humidifier is configured to diffuse hydrogen peroxide into the air. In some embodiments, the Peroxide Humidifier is configured to diffuse about 450 ml/hr hydrogen peroxide into the air. The diffusion rate is set by the mechanical structure of the evaporator device surface area and the choice of fan air flow design to exactly match the grams per hour output of the ozone generator device in order to achieve maximum production of Hydroxyl radicals. The vaporization method can be a soak able media like a cloth mesh or open cell foam. This media can be wetted by various methods including water wall flooded gravity method or a spray method involving a nozzle jet. Either method requires a liquid feed rate greater than the desired rate of 450 ml/hr vaporization rate. The 450 ml/hr rate is determined using standard 3% concentration solution available in any pharmacy or drug store. Other combinations of higher concentrations and lower flow rates can be used to achieve the same results. The ideal range is 300 to 500 ml/hr using 3% solution. Higher concentrations of hydrogen peroxide can be used but require special handling and can be hazardous if not handled properly. The operation temperature for the Humidifier is 4C to 32C but ideally it would be used at 25C for best predictive treatment results. In some embodiments, the Peroxide Humidifier is configured to diffuse about 453 ml/hr hydrogen peroxide into the air. In some embodiments, the Ozone Generator and the Peroxide Humidifier are configured to communicate status information. In some embodiments, the Ozone Generator and the Peroxide Humidifier are configured to communicate through a wireless connection. In some embodiments, the Ozone Generator and the Peroxide Humidifier are configured to communicate through a wired connection. In some embodiments, at least one of the Ozone Generators and the Peroxide Humidifier includes a sensor and is configured to communicate sensor information. In some embodiments, at least one of the Ozone Generators and the Peroxide Humidifier is electrically connected to communication circuitry external thereto. In some embodiments, at least one of the Ozone Generators and the Peroxide Humidifier weighs less than 15 lbs. The 15 lbs. limit is dictated by the OSHA human factors regulation upper limits for a device and the prevention of injury to the user transporting the device. In some embodiments, the system further includes an Equipment Control Device, configured to communicate with at least one of the Ozone Generators and the Peroxide Humidifier. In some embodiments, the Worksite Control Device is further configured to communicate with another duplicate Worksite Control Device. In some embodiments, the Worksite Control Device is further configured to communicate with equipment located in a building remote to the system or in a service vehicle. In some embodiments, the Worksite Control Device is located within a device located outside the treatment area. In some embodiments, the Worksite Control Device communicates via a local cellular network, a wide-area network, or the internet. In some embodiments, the Worksite Control Device has a uniform resource locator (URL). In some embodiments, the Worksite Control Device has a short-range antenna, and a connection for an external extended range antenna. In some embodiments, the Worksite Control Device has one or more connections for at least one of a display, a keypad, a pointing device, a printer, and other equipment. In some embodiments, the Worksite Control Device is configured to operate on battery power for at least 12 hours without recharging. In some embodiments, the Worksite Control Device is portable. In some embodiments, the Worksite Control Device weighs less than 15 lbs. In some embodiments the Worksite Control Device is in a fixed location as part of a building.

In another aspect, an Ozone Generator device includes a housing, and a lamp within the housing, where the lamp is configured to emit ozone generating light. The device also includes a plurality of walls, including openings therein, where the openings form a plurality of passages for ozone generated by the light to escape from the housing, and the walls prevent the light from escaping from the housing.

In some embodiments, the device further includes a sensor within the housing. In some embodiments, the device weighs less than 15 lbs. In some embodiments, the device further includes a connection for communications circuitry. In some embodiments, the connection includes a wireless connection. In some embodiments, the connection includes a wired connection. In some embodiments, the connection includes a USB or a firewire, fiber optics, haptic communication. Acoustic coupling and visual optical transmitters and receivers can be substituted as communications media in place of a wire conductor. In some embodiments, the device further includes communications circuitry. In some embodiments, the communications circuitry includes circuitry for wireless communication. In some embodiments, the communications circuitry includes circuitry for wired communication. In some embodiments, the device further includes a sensor, where the communications circuitry is configured to communicate sensor information. In some embodiments, the communications circuitry is configured to communicate status information. In some embodiments, the device further includes a microphone connected to the housing. In some embodiments, the device further includes a speaker connected to the housing. In some embodiments, the communications circuitry is configured to communicate with hardware, a controller, or an individual. In some embodiments, the communications circuitry is configured as a node on a communications network including wired and wireless communication links. In some embodiments, the communications take place over NFC, Wi-Fi, or Bluetooth protocols. In some embodiments, the communications circuitry includes a processor and memory. In some embodiments, the processor and the memory are configured to operate a local operating system and one or more software applications. In some embodiments, one of the software applications is configured to process data to generate a result and the communications circuitry is configured to communicate the result. In some embodiments, the acquired sensor information is stored in a data lake where it is data mined for information. The data lake is accessible from a user interface mobile device held by a technician. In another embodiment the user interface enables access control to all hardware performing the hydroxyl generation process. In some embodiments, the communications circuitry is configured to operate with power from at least one of a building, an automobile power, generator, engine power, and a self-contained rechargeable or single use battery. In some embodiments, the communications circuitry is uniquely identifiable. In some embodiments, the device is configured to output about 10 grams/hour of ozone. In some embodiments, the light has a first wavelength below about 200 nanometers. In some embodiments, the light has a first wavelength of about 185 nanometers. In some embodiments, the light has a second wavelength of about 254 nanometers. In some embodiments, the openings in the walls are staggered and where a ratio of 185 nm energy to 254 nm energy is about 1. In some embodiments, the lamp includes a mercury vapor lamp. In some embodiments, the lamp is in a controlled air flow environment of about 200 cfm. In some embodiments, the lamp includes extra heavy-duty tungsten conductors configured to minimize damage from shock and vibration. In some embodiments, the lamp includes an 800 ma, 96-watt mercury lamp. The power range can be from 80 to 100 watts and the lamp current can range from 50 to 150 watts depending on lamp pressure and ballast efficiency. In some embodiments, the lamp includes a quartz u-tube about 17 inches in length. In some embodiments, the walls include aluminum. In some embodiments, the walls include a material which absorbs the light from the lamp. In some embodiments, the walls include a coating which absorbs the light from the lamp. In some embodiments, the lamp is mounted to the housing with a fluorosilicone isomer. Other floroelastomer compounds can be used as substitutes if the compounds resist 100 to 300 nanometer light sources and temperatures of 200C.

In another aspect, a method of treating air includes providing an Ozone Generator with communication circuitry, providing a Peroxide Humidifier with communication circuitry, sending a first signal from the Ozone Generator to the Peroxide Humidifier, and sending a second signal from the Peroxide Humidifier to the Ozone Generator. In some embodiments, the Peroxide Humidifier and the Ozone Generator send signals to an Equipment Control Device. In some embodiments, the first signal includes status information. In some embodiments, the second signal includes status information. In some embodiments, the method further includes diffusing hydrogen peroxide into the air with the Peroxide Humidifier. In some embodiments, the method further includes diffusing about 450 ml/hr hydrogen peroxide into the air with the Peroxide Humidifier. In some embodiments, the method further includes diffusing about 453 ml/hr hydrogen peroxide into the air with the Peroxide Humidifier. In some embodiments, the method further includes outputting about 10 grams/hour of ozone with the Ozone Generator. In some embodiments, both the hydrogen peroxide and ozone generation embodiments interact to generate hydroxyl radical formation. In some embodiments sending the first signal includes sending the first signal through a wireless channel. In some embodiments, sending the first signal includes sending the first signal through a wired channel. In some embodiments, at least one of the Ozone Generators and the Peroxide Humidifier includes a sensor, and at least one of the first and second signals includes sensor information. In some embodiments, the method further includes sending a third signal from at least one of the Peroxide Humidifiers and the Ozone Generator to a Worksite Control Device. Each device is capable of communicating with other devices in the system, either directly, or through another device. In some embodiments, the method further includes sending a fourth signal from the Worksite Control Device, a second Worksite Control Device of a separate system. In some embodiments, the method further includes sending a fifth signal from the Worksite Control Device to equipment located in a building remote to the system or in a service vehicle. Some embodiments include the use of wireless mesh networking technologies over Wi-Fi, Bluetooth and/or other protocols for better overall wireless communications range between devices. In some embodiments, the method further includes emitting ozone generating light with a lamp within the Ozone Generator. In some embodiments, the method further includes sending a sixth signal from one of the Peroxide Humidifiers and the Ozone Generator to a controller or to an individual via a communication device, e.g., speaker, display, handheld device, smartphone, computer, etc. In some embodiments, at least one of the Peroxide Humidifiers and the Ozone Generator includes a processor and a memory, and the method further includes the processor and the memory operating a local operating system and one or more software applications. In some embodiments, one of the software applications is configured to process data to generate a result and one of the first and the second signals includes the result. In some embodiments, the method further includes operating the Peroxide Humidifier and the Ozone Generator with power from at least one of a building, an automobile power, and a self-contained rechargeable battery.

In another aspect, a method of manufacturing an Ozone Generator device includes providing a housing and mounting a lamp within the housing, where the lamp is configured to emit ozone generating light. The method also includes connecting a plurality of walls to the interior of the housing, where the walls include openings therein, and where the openings are configured to allow ozone generated by the light to escape from the housing and to prevent the light from escaping from the housing. A number of methods to accomplish this include diffusion layers, alternate hole patterns on multiple layers of material, bent pipe between the light source and the ozone exit.

Additional operator information on functionality can be gained by lamp indicators on the top side of the ozone generation units. Other methods of indicators can be used including matrix displays, reflective indicators, light pipes or mechanical thermal or movement indicators.

An operating hours clock mechanism indicates total operating time on the ozone generation units. A clock mechanism is needed for maintenance information on a specific unit where electronic run time may not be available or may differ due to additional testing cycles not tracked by our electronic run time indicators from treatment cycles.

A typical ultrasonic vaporizer commercially available is designed to vaporize water. The corrosive nature of hydrogen peroxide in a hydroxyl atmosphere requires more inert packaging materials construction to be used for treatments. In addition, typical ultrasonic vaporizers lack the spill prevention and monitoring ability needed for treatments. In another aspect, a Peroxide Humidifier includes a housing enclosing a first chamber and a second chamber, the first chamber holding a hydrogen peroxide solution, and the second chamber coupled to an ultrasonic mechanism configured to vaporize hydrogen peroxide in the second chamber. The second chamber is in fluid communication with the first chamber and the second chamber is in fluid communication with the ambient atmosphere such that vaporized hydrogen peroxide can enter the atmosphere. By employing this method, the vapor created by the ultrasonic device can be diffused into the air preventing large droplets from leaving the device and contacting surfaces. This method also prevents spillage if the unit is tipped over. The two chamber method allows vapor to escape but contains liquid against spillage if lying on its side.

Additional operator information is available on Peroxide Humidifier units. A visual tank depth viewing slot with optional backlighting button available before and after treatments on an independent power system. This visual indicator performs a setup verification prior to a treatment and electronic indication of depth.

In some embodiments, the hydrogen peroxide solution is isolated from the outside when the Peroxide Humidifier is upended, inverted, or left on its side. This is accomplished by never filling peroxide tanks more than half full and installing atmospheric venting tubes on the tanks crossing from side to side preventing tank leakage beyond 10 milliliters when rotated in any orientation and left on any of 6 sides.

In another aspect, a Peroxide Humidifier includes an air inlet, an air outlet, an evaporator, and a fan configured to force air from the air inlet through the evaporator and out of the Peroxide Humidifier through the air outlet. The Peroxide Humidifier also includes a chamber configured to hold a solution, a pump, and a fluid circulation path including the pump, the chamber, and the evaporator, where at least a portion of the solution exits the evaporator in the air forced through the evaporator. The pump in this application needs the ability to start suction, draw from a fluid reservoir, and be inert enough to withstand the corrosive nature of the chemistry involved in a treatment cycle. It also needs to be small and light weight with Teflon, Santoprene, or fluoropolymer elastomer components.

In some embodiments, the evaporator includes a diffusion layer, configured to allow the air to flow therethrough and to allow the solution to flow therethrough, where at least a portion of the solution exits the diffusion layer in the air, and first and second air permeable layers on opposite sides of the diffusion layer. In some embodiments, the evaporator further includes first and second screens, where the first air permeable layer is compressed between the first screen and the diffusion layer, and the second air permeable layer is compressed between the second screen and the diffusion layer. Other embodiments can include sponge materials, inert fibrous material, open cell foam or wettable screen configuration materials.

In another aspect, an Ozone Generator device includes a housing, including an air inlet, an air outlet, and a lamp within the housing, where the lamp is configured to emit ozone-generating light. The lamp includes a glass enclosing a gas, one or more pins configured to electrically connect to a power source, a filament within the glass, and first and second electrodes extending through the glass and electrically connected to the pin. The filament is electrically and mechanically connected to the first and second electrodes. The Ozone Generator also includes a fan configured to force air from the air inlet out of the device through the air outlet, where the air exiting the device includes ozone. The fan configuration can include centrifugal devices, bladeless ultrasonic or electrostatic, impellers, propellers, or other energy driven devices.

In some embodiments, the filament is medium gauge. Light gauge is known to be 10 to 40 micrometers in diameter, medium gauge being 40 to 300 micrometers in diameter. In some embodiments, the filament includes first and second ends and the filament is connected to the first and second electrodes at the first and second ends, respectively. This configuration prevents breakage and medium gauge can be used because the required internal temperature required to keep a discharge tube operating is lower than an incandescent application and heavier filament can be used to prevent breakage in rough handling situations.

In another aspect, a method of introducing a solution into air includes providing a Peroxide Humidifier including an air inlet and an air outlet, pumping a solution from a storage chamber to an evaporator, collecting the solution from the evaporator in the storage chamber, and propagating air from the air inlet through the evaporator to the outlet, where a portion of the solution enters the air in the evaporator and exits the Peroxide Humidifier with the air through the air outlet.

In some embodiments, the method further includes forcing air to flow through a diffusion layer in the evaporator, and providing solution to flow through the diffusion layer, where a portion of the solution exits the diffusion layer with the air. In some embodiments, pumping the solution includes pumping the solution to a top of the evaporator, where the evaporator is vertically oriented. In some embodiments, propagating the air includes forcing the air with a fan. In another embodiment, the diffusion layer is configured to guide vertical water addition to the lower point keeping the flow in the center of the diffusion layers in the event the evaporator is tilted on axis during operation.

### Detailed Description of the Preferred Embodiment

The following description and examples illustrate some exemplary embodiments of certain inventive aspects. Those of skill in the art will recognize that there are numerous variations and modifications that are encompassed by the scope of the disclosure and the appended claims. Accordingly, the description of any particular exemplary embodiment should not be deemed to limit the scope of the present description.

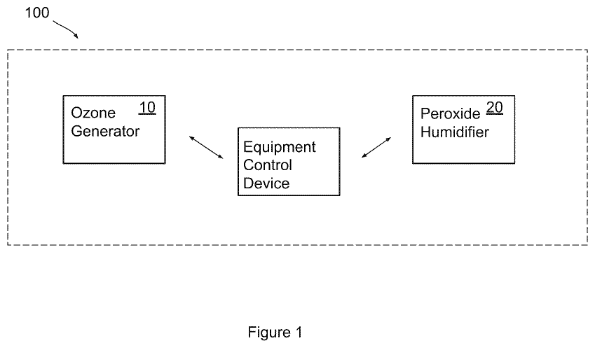

FIG. 1 is a block diagram of a system 100 used for treating the air and all exposed surfaces within the treatment area. The system 100 includes an Ozone Generator 10 and a Peroxide Humidifier 20. In some embodiments the Peroxide Humidifier 20 is configured to diffuse hydrogen peroxide into the air. The Ozone Generator 10 and the Peroxide Humidifier 20 are configured to cooperatively operate to treat air. The system 100 may be configured to treat the air and all exposed surfaces within the treatment area according to methods described in U.S. Pat. No. 7,407,624, which disclosure is incorporated herein by reference in its entirety. In some embodiments the Ozone Generator 10 and Peroxide Humidifier 20 have switches, hour meters, and/or an additional plug for other equipment, such as other Ozone Generators, Peroxide Humidifiers, and fans.

In some embodiments the Ozone Generator 10 and Peroxide Humidifier 20 have communications circuitry capable of communicating with each other and/or with other equipment. One such embodiment is discussed further below. In some embodiments the communications circuitry is integrated with the Ozone Generator 10 and the Peroxide Humidifier 20. In some embodiments the communications circuitry is included in a separate piece of equipment and communicates with at least one of the Ozone Generators 10 and the Peroxide Humidifier 20 through a wire, such as a USB connection, firewire or other wired standard computer interface connections. In some embodiments the communications circuitry communicates wirelessly with at least one of the Ozone Generators 10 and the Peroxide Humidifier 20. In some embodiments the Ozone Generator 10 and the Peroxide Humidifier 20 may be configured to communicate status information, sensor information and/or verification information with each other and/or with other equipment. In some embodiments the Ozone Generator 10 and the Peroxide Humidifier 20 communicate with one another. In some embodiments the Ozone Generator 10 and the Peroxide Humidifier 20 additionally or alternatively communicate with other equipment. In some embodiments one or both of the Ozone Generator 10 and the Peroxide Humidifier 20 include environmental, video and/or audio sensors. In some embodiments environmental, video and/or audio sensors, and/or the communication capabilities of the Ozone Generator 10 or the Peroxide Humidifier 20 may be used to communicate with hardware, a controller, or an individual. The hardware may be, for example, part of a network or a stand-alone device. The controller may be, for example, a local or a remote device. Some embodiments use temperature sensors on the inlet and outlet of the Peroxide Humidifier to determine vaporization rate and room humidity conditions. Some embodiments show sensors for hydrogen peroxide availability during operation. Some embodiments show radiant energy available through lamp current sensors during operation. Lamp current is an indication of the lamp's contribution to the production of hydroxyl radicals in combination with software running on processors within the system. The production of hydroxyl radicals can be controlled under command of the ozone and hydrogen peroxide transit cases and software resident on controllers and a data lake. An embodiment of an operator interface, data storage, transit case subsystems and controllers running code make up a treatment process.

In some embodiments the communication circuitry serves as a node on a communications network comprising wired and wireless communication links. Each communication circuit may include, for example, a processor with local memory capable of operating on household current, generator power and/or a self-contained rechargeable or single use battery. The communication circuit may also have network adapters, antennas and/or connectors to conform to the communications network protocols. The processor may have a local operating system and have provisions for custom software applications. Each communications circuit may be uniquely identifiable. In addition, the communications circuits may interface with either the Ozone Generator or the Peroxide Humidifier equipment and receive or sense the operating state of the associated equipment (for example, powered-on or powered-off). Local processing of data by the communications circuitry may be performed. This may minimize the amount of data transmitted from or received by the communications circuitry.

The Ozone Generator 10 and the Peroxide Humidifier 20 each may be designed to weigh less than 15 lbs. In some embodiments the Ozone Generator 10 and/or the Peroxide Humidifier 20 may be housed in a case having rounded corners and formed of a deformable material to minimize worksite area damage, survive transit environment, and withstand the continual exposure to high concentrations of oxidants such as the hydroxyl radicals generated in the process. In some embodiments the Peroxide Humidifier 20 diffuses about 450 ml/hr hydrogen peroxide into the air. For example, the Peroxide Humidifier 20 may diffuse about 453 ml/hr hydrogen peroxide into the air using a storage containing a 3% hydrogen peroxide solution. In some embodiments ultrasonic energy is used to create micro particles which are then diffused into a 200 cfm air stream.

The Ozone Generator 10 outputs up to and including about 20 grams/hour of ozone using custom lamp technology. For example, to generate ozone with a lamp, a gas within the lamp is ionized by a current. The excited gas emits an electromagnetic energy having a wavelength below about 200 nanometers. The electromagnetic energy transforms O₂ into O₃.

In some embodiments a mercury vapor lamp may be used. The ionized mercury vapor has two emission peaks, one at about 254 nm and one at about 185 nm. In some germicidal UV lamps, only the 254 nm peak is detectable unless the outer lamp material is made of fused quartz. Another aspect is a lamp may produce energy emission ratios of about 1 unit of 254 nm energy for every ¼ unit of 185 nm energy. However, the 254 nm energy and the 185 nm energy do not cooperatively generate O₃. The 185 nm emissions transform 3 O₂s into 2 Oss, but the 254 nm emissions destroy O₃ by transforming 2 O₃s into 3 O₂. Accordingly, it is desirable to adjust the generation environment such that more O₃ is generated and less O₃ is destroyed. In some embodiments the Ozone Generator 10 also incorporates thermal and current fuses to cut power in the event of equipment failure.

One feature for producing more O₃ increases the ratio of 185 nm emissions to 254 nm emissions. This can be accomplished, for example, by adjusting the temperature of the ionized gas. Lamp current for a mercury vapor lamp can be selected to manipulate a ratio of emissions. As the ionized gas temperature rises, the emission level of the 254 nm energy decreases while the 185 nm emission remains substantially constant. Accordingly, a higher temperature results in an increased ratio of 185 nm emissions to 254 nm emissions. In some embodiments the ratio of 185 nm energy to 254 nm energy is about 1. This results in higher levels of ozone production when compared to conventional methods. In some embodiments, the temperature is preferably between about 100° F. and about 200° F.

In some embodiments the lamp is placed in a controlled air flow environment in order to prevent lamp breakage. For example, about 200 cfm of air flow over the lamp may be used to prevent overheating and breakage. In some embodiments the inside temperature is monitored by analog integrated circuits. In addition, hall effect current sensors may be used to monitor total lamp power and functionality with a plurality of lamps in one location. Additionally, or alternatively, because the increased temperature leads to increased pressure in the lamps, extra heavy-duty tungsten conductors may be used with the end filaments. The heavy-duty conductors may be configured to withstand the extra power and mechanical shock. In some embodiments 800 ma (96 watts) mercury lamps with extra heavy duty tungsten conductors are used. In some embodiments the tungsten filaments are formed with an oversized gauge when compared to conventional lamps to accommodate the additional current. This also gives much better resistance to mechanical shock. In addition, the filaments may be designed for 4 wire ballasts, but the lamps themselves may be 2 wire. The additional second support secures the filament from both ends similar to a conventional light bulb design, which further increases the shock and vibration rating needed for rough transport portable equipment. In some embodiments the lamps are custom made for the Ozone Generator 10. In some embodiments the lamp envelopes are made of heavy wall quartz u-tubes of about 17 inches in length.

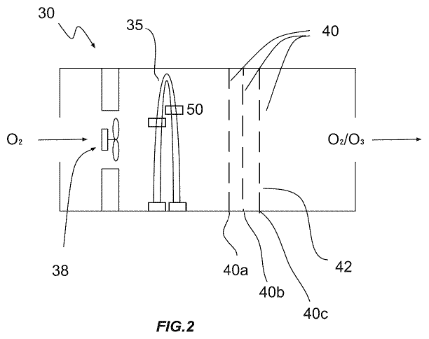

Another aspect is a second feature for producing more O₃ reduces the amount of time the O₃ is exposed to 254 nm emissions. FIG. 2 is a schematic diagram illustrating an Ozone Generator 30 which can be used in system 100 of FIG. 1. The Ozone Generator 30 has features for reducing the amount of time the generated O₃ is exposed to 254 nm emissions. The Ozone Generator 30 may include a fan 38, a lamp 35 and walls 40. The walls 40 may be opaque to the lamp light and have openings 42, which allow the generated O₃ to exit. Because of the Venturi effect, the pressure at the openings is lower than the pressure at the fan 38. Once the O₃ passes through the openings 42 of the first wall 40a, the O₃ is shaded from the O₃ destroying 254 nm emissions from the lamp by the non-opening portions of the first wall 40a. Likewise, once the O₃ passes through the openings 42 of the second wall 40b, the O₃ is shaded by the non-opening portions of the first and second walls 40a and 40b from the 254 nm emissions. In some embodiments once the O₃ has passed the last wall 40c, the O₃ is completely shaded from the lamp 35 by the walls 40. In some embodiments more than three walls are used. In some embodiments fewer than three walls are used. In some embodiments multiple walls having slits of differing orientation are used. Additional beneficial aspects of this arrangement can include that the light from the lamp, which light can be harmful to people, does not escape the Ozone Generator 30 due to the staggered configuration of openings 42 in the first wall 40a, the second wall 40b and the third wall 40c. In some embodiments the walls 40 may include aluminum. In some embodiments the walls 40 may include a material which absorbs the light from the lamp and minimizes reflection. In some embodiments the walls 40 may include an oxide coating.

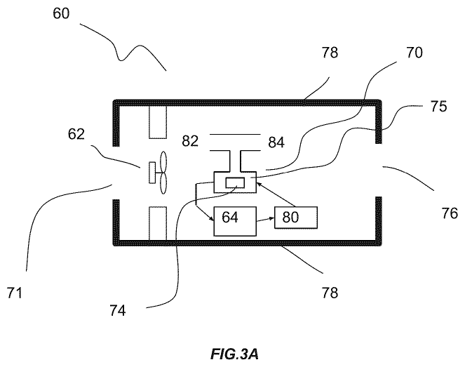

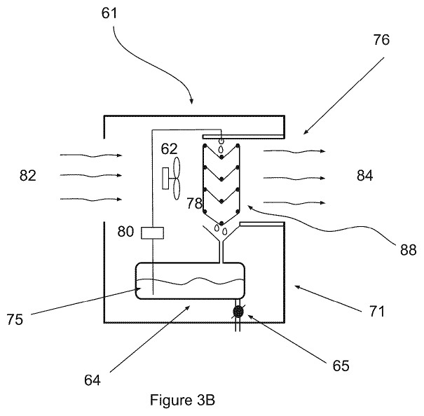

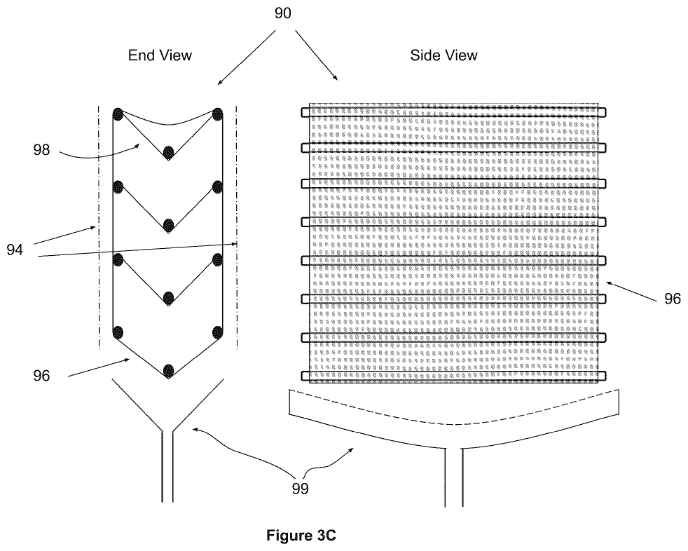

To protect the lamp 35 from mechanical shock, the lamp 35 may be mounted to the Ozone Generator 30 with a fluorosilicone isomer 50 in four places. The mount 50 may be effective in absorbing mechanical shock and may be resistant to the corrosive effects of the environment due to any of the ozone, the light and/or the hydrogen peroxide. In another embodiment, lamps may be mounted with spring wire of a non-corrosive material to minimize shock, FIG. 3A 50.

FIG. 3A is a schematic diagram illustrating a Peroxide Humidifier 60 which can be used in system 100. The Peroxide Humidifier 60 includes a case 71, a fan 62, air paths 82 and 84, a primary storage chamber 64, an ultrasonic vaporizer chamber 70 containing solution 75, an ultrasound source 74, a pump 80. Another aspect is a water and hydrogen peroxide solution 75 is stored in the primary storage chamber 64.

The fan 62 forces air through the case 71 which exits through the outlet 76. At least a portion of the forced air travels through air path 82 into the ultrasonic vaporizer chamber 70, where it mixes with diffused hydrogen peroxide, before continuing through air path 84, and out the outlet 76.

The water and hydrogen peroxide solution is provided to the storage chamber 64 through a filler tube (not shown), while gas escapes through a vent (not shown). The solution is introduced to the ultrasonic vaporizer chamber 70 with pump 80. In some embodiments, the depth of the solution within the vaporizer chamber 70 is maintained substantially constant. For example, the depth may be maintained at about 1 inch, about 1.5 inches, or about 2 inches. In some embodiments, the overflow path 86 maintains the level substantially constant. The overflow path 86 may be gravity driven and in some embodiments, includes multiple paths. In some embodiments, at least one overflow path 86 is smaller than another overflow path 86. For example, the smallest of the overflow paths 86 may be sized so that it is substantially filled with solution returning from the vaporizer chamber to the storage chamber 64. In some embodiments, the overflow path 86 comprises paths which join before connecting with the storage chamber 64. For example, overflow path 86 may include two tubes which are each connected to the vaporizer chamber 70, where one tube is ¼ inch tubing and the other is 1/16 inch tubing. The 1/16 inch tube may join the ¼ inch tube with a “T” junction, from which the overflow path 86 continues with a ¼ inch tube from the “T” junction to the storage chamber 64.

Within the chamber 70, the solution is vaporized by the ultrasound source 74. The ultrasound source 74, may be, for example, an about 20-40 KHz ultrasound device. The ultrasonic energy from the ultrasound source 74 causes a portion of the solution to vaporize. The air flow through air paths 82 and 84 carries the vaporized solution out of Peroxide Humidifier 60 through outlet 76.

Spillage of the hydrogen peroxide solution can cause damage to other items, such as carpets and furniture. The Peroxide Humidifier 60 may be configured to effectively inhibit spillage of the hydrogen peroxide solution when the portable equipment is upended, inverted, or left on its side. To accomplish this, the storage chamber 64, the vaporizer chamber 70, and one or more additional storage chambers are in fluid communication and include sufficient capacity that when the portable equipment is upended, inverted, or left on its side the solution does not flow into the air paths 82 and 84. For example, for 1 gallon of solution at least an additional 1 gallon of chamber capacity may be used. In some embodiments the primary storage chamber 64 holds approximately 1 gallon of the hydrogen peroxide solution, thus limiting the possible spillage therefrom to approximately 1 gallon. In some embodiments the depth can be verified by a depth sensor located in the storage chamber. In some embodiments, the vaporizer chamber 70 is sized and shaped so that if the Peroxide Humidifier 60 is upended, inverted, or left on its side, the level of the solution is isolated from the entrance to the air paths 82 and 84 and is therefore isolated from the outside. In some embodiments, a third chamber (not shown) is in fluid communication with the storage chamber 64 and the vaporizer chamber 70, such that if the Peroxide Humidifier 60 is upended, inverted, or left on its side, the level of the solution is isolated from the entrance to the air paths 82 and 84, and is therefore isolated from the outside. In some embodiments, there is a gap between each of the air paths 82 and 84 and the case 71, so that even if solution escapes from the vaporizer chamber 70, the solution remains within the case 71, and is adsorbed by adsorption media 78 within the case 71. In some embodiments, adsorption media 78 is configured to absorb spillage and will renew itself during transit or subsequent operation by slowly releasing adsorbed solution.

FIG. 3B is a schematic diagram illustrating a Peroxide Humidifier 61 which can be used, for example, in system 100. The Peroxide Humidifier 61 includes a case 71, a fan 62, air paths 82 and 84, an evaporator 88, a primary storage chamber 64 containing solution 75, a pump 80, and absorption media 78. Another aspect is a water and hydrogen peroxide solution 75 is stored in the primary storage chamber 64.

The fan 62 forces air through the case 71 which exits through the outlet 76. At least a portion of the forced air travels through air path 82 to the evaporator 88, where it mixes with hydrogen peroxide. The air continues through air path 84 and out the outlet 76.

Solution 75 is pumped by pump 80 from the storage chamber 64 to evaporator 88. The solution 75 flows through evaporator 88, where a portion of the solution 75 evaporates or diffuses into the air flowing through the evaporator 88. In some embodiments, the solution 75 is pumped through the evaporator 75 by the pump 80. In some embodiments, the solution flows through evaporator 75 because of gravitational pull. The solution 75 flows from the evaporator 75 to the storage chamber 64.

FIG. 3C is a schematic diagram illustrating an evaporator 90, which is an embodiment of an evaporator, which may be used, for example, in the Peroxide Humidifier 61 of FIG. 3B. Evaporator 90 includes screens 94, air permeable layers 96, and diffusion layer 98. In this embodiment, the air flows from one vertical side of the evaporator 90 to another. Air flows into the evaporator through a first screen, and continues through a first air permeable layer 96, through the diffusion layer 98, through a second air permeable layer 96, and out of the evaporator 90 through a second screen 94.

Diffusion layer 98 is configured to allow the solution 75 to flow therethrough. In this embodiment, the solution 75 flows from top to bottom due to gravitational pull. As discussed above, the diffusion layer 98 is configured to allow air to flow therethrough. As the air passes through the diffusion layer 98, it mixes with a portion of the solution 75 to achieve or maintain a desired concentration of the solution 75 in the air. The diffusion layer 98 is substantially inert to the solution 75. The diffusion layer 98 is absorbent so that it may function to wick up the solution. In some embodiments, the diffusion layer 98 may include, for example, at least one of cotton, fiberglass, and polyethylene fabric. In some embodiments, the diffusion layer 98 is formed about 1/16 inch thick.

In this embodiment, the diffusion layer 98 is sandwiched by air permeable layers 96. The air permeable layers 96 are configured to allow air to flow therethrough both to and from the diffusion layer 98. In addition, the air permeable layers 96 may provide mechanical support to the diffusion layer 98 as a result of the air permeable layers 96, which may be mechanically compressed between one of the screens 94 and the diffusion layer 98. Some of the mechanical support for the diffusion layer 98 coming from outside the diffusion layer 98 may allow for optimization of solution flow and air flow properties of the diffusion layer 98 while easing mechanical structure constraints. In some embodiments, the air permeable layers 96 are formed about ½ inch thick. In some embodiments, the permeable layers 96 comprise at least one of polypropylene and polyethylene fiber material having fibers with diameter about 0.005 inches.

The screens 94 allow air to flow to and from the air permeable layers 96 and provide mechanical support for the air permeable layers 96. In some embodiments, the screen 94 is a ½ inch by ½ inch grid. In some embodiments, the screens 94 include metal.

In some embodiments, the evaporator 90 has dimensions of about 6 inches by about 7 inches by about 1¼ inches. The amount of solution 75 which is provided to the evaporator 90 is determined so that solution 75 is available for gaseous diffusion into the air, and so that air flows through the evaporator without forcing large amounts of liquid solution, or in some embodiments, any liquid solution out of the evaporator 90.

In some embodiments, the evaporator 90 includes multiple diffusion layers 98, each of which is sandwiched by air permeable layers 96. Add something on layer layout to survive use in a non-flat environment. In some embodiments, 99 is configured to allow operation in non-level surfaces without leakage. 98 also serves to steer water droplets to allow operation on non-level surfaces.

In some embodiments high pressure misting heads targeted at a drain back system allow the proper sized droplets to be picked up and carried away with the air stream. In some embodiments the droplets generally range in size from about 1 μm to about 10 μm with an average of about 5 μm for an ultrasonic method and range from about 10 μm to about 1000 μm for a spray method. For the spray method the water surface area may be significant. One advantage of using the misting heads may include weight savings, which are possible because the power conditioning requirements to run low voltage high power ultrasonics can be eliminated. Another advantage of using the misting heads is that the maximum humidity levels in the room can be automatically regulated because the system meters less in humid conditions and more in dry conditions, such as at start up. In some embodiments regulation is accomplished automatically by vapor deficit calculations from 100%, such that dryer air results in a greater evaporation rate. Temperature sensors on the inlet and outlet allow for evaporation and humidity monitoring.

In some embodiments the Ozone Generator 10 and/or Peroxide Humidifier 20 of FIG. 1 also include one or more condition monitoring sensors. The sensors may be used to monitor, for example, ozone concentrations, peroxide concentrations, humidity, temperature and/or air flow. In some embodiments sensors monitoring the status of the Ozone Generator 10 or Peroxide Humidifier 20 can be used. For example, a sensor may monitor the output level of the Ozone Generator 10 or Peroxide Humidifier 20, and/or whether the Ozone Generator 10 or Peroxide Humidifier 20 is operating within specification. In some embodiments the sensors include one or more cameras configured to generate still images or video images. In some embodiments the sensors include one or more microphones. Other embodiments for a full sensor suite of sensors are not limited to thermal, G-force, motion, spatial coordinates, rotation, atmospheric conditions to ensure a safe treatment process.

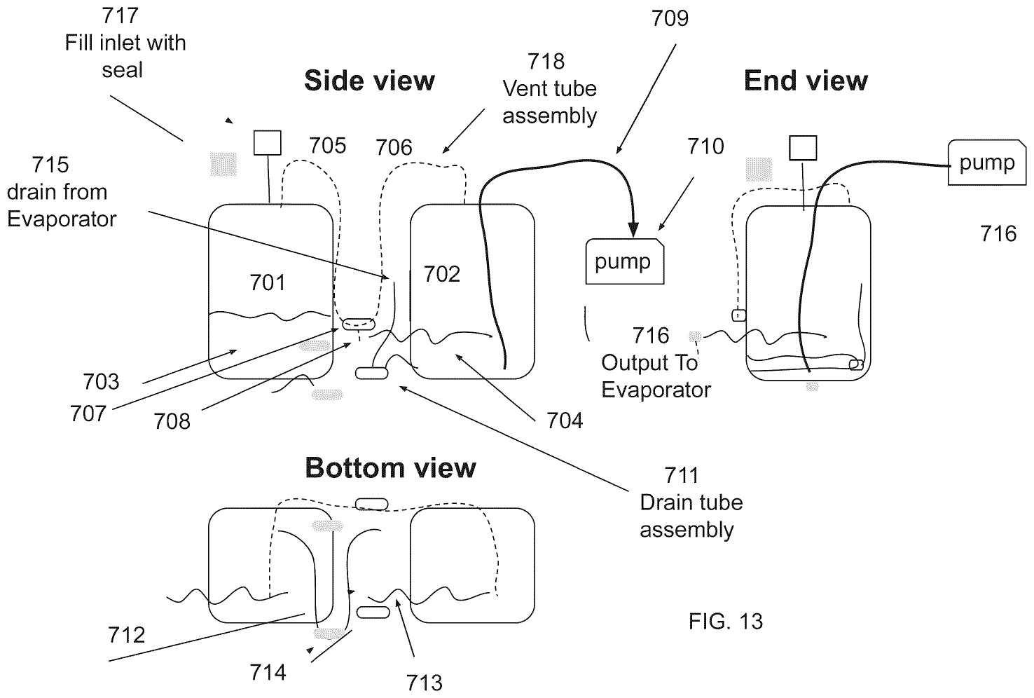

A fluid retention embodiment can be used in the portable Peroxide humidifier in the event the portable transit case is inverted, upended, or stored in one of five surface orientations not in the typical vertical orientation for a transit case. FIG. 13 depicts the system to manage the fluid in the retention in tanks in a manner as to prevent leakage from the tanks beyond minor drippage. 700 is the fluid management system shown in three views describing the retention system. Tanks 701 and 702 have a holding capacity of 1-gallon combined capacity. Peroxide liquid levels 703 and 704 are below 50% at the full condition. 717 represent the filler tube and inlet seal assembly to allow fluid addition from the top of the tank. Transfer of liquid between tanks 701 and 702 may occur through the drain tube assembly 711. The drain tube consists of 3 parts, S tube 712 running between the bottom corner of tank 701 to a Tee fitting 714. From the Tee fitting 714 a second S tubing 713 is run over to the corner of tank 702. The third connection of the Tee fitting 714. Tubing 715 rises above the fluid level in tanks 701 and 702 and receives drainage from the evaporator 78 in FIG. 3B. Tubing 709 draws peroxide 703 from tanks 701 and 702 and enters a positive displacement pump 710 (item 80 in FIG. 3B) and moves peroxide to the top of the evaporator (item 78 FIG. 3B) by way of tubing 716. Pumping peroxide from tank 701 and 702 creates a negative pressure requiring vent assembly 711 to balance the internal tank pressure with that of the atmosphere surrounding the tank. The vent assembly 711 requires S tubing 705 to run from the top of the back wall of the center line of tank 701 and down the front surface of tank 701 to a Tee fitting 707. S tubing 706 is connected to the top back wall of the center line of tank 702 and down the front surface of tank 702 to a Tee fitting 707. The third Tee connection 703 is open to the atmosphere by vent 708. The location of vent 708 and Tee fitting 714 form a fluid lock in upended orientations by the drain assembly 711 and the vent tube assembly 718 reversing roles as the peroxide fluids held to the lower portion of any tank by gravity as the tanks 701 and 702 are repositioned by rotation. Once repositioned to a new orientation, material in the S tubes tube may lose contents to drippage but tanks 701 and 702 will remain filled with peroxide solution. Minor drippage will be absorbed by internal wicking material for this purpose.

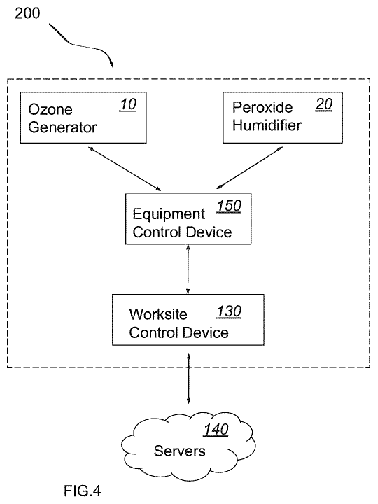

FIG. 4 depicts a schematic diagram of system 200 used for treating the air and all exposed surfaces within the treatment area. The system 200 includes an Ozone Generator 10, a Peroxide Humidifier 20, and a Worksite Control Device 130. The Ozone Generator 110 and the Peroxide Humidifier 120 are configured to communicate with an Equipment Control Device 150 and Worksite Control Device 130 and may, for example, be similar to Ozone Generators and Peroxide Humidifiers discussed above. The system 200 is also configured to communicate with other equipment 140. The other equipment 140 may be located at, for example, a central office or a field technician's vehicle or any location separate from the treatment area. Thus, in some embodiments the Ozone Generator 110, the Peroxide Humidifier 120 and/or the Equipment Control Device 150 and Worksite Control Device 130 can communicate with computing equipment indicated in 140.

The Worksite Control Device 130 is capable of communicating with other Worksite Control Devices and/or communications circuits, and/or with other equipment 140. In some embodiments the Worksite Control Device 130 is located within a lockable box which is securely located on the outside of a building being serviced. In some embodiments the lockable box has an attachment member configured to connect the box to a standard doorknob. In some embodiments the Worksite Control Device 130 has similar capabilities and features as the communications circuits described above. In addition, the Worksite Control Device 130 serves as an interface between local communications circuits operating at a service site and the other equipment 140. In some embodiments the Worksite Control Device 130 communicates via a local cellular or other wide-area network. In some embodiments the Worksite Control Device 130 communicates via the internet. In some embodiments the Worksite Control Device 130 has a uniform resource locator (URL). The Worksite Control Device 130 may be located on a service vehicle, such as a truck parked within communications range of the communication circuits at the service site. In some embodiments in addition to a short-range antenna, the Worksite Control Device 130 has a connection for an external extended range antenna. The Worksite Control Device 130 may also have connections for a display, a keypad, a pointing device, a printer, and/or other equipment. In some embodiments the Worksite Control Device 130 is capable of operating on battery power for up to 12 hours without recharge or connection to external power sources. In some embodiments the Worksite Control Device 130 is portable and weighs less than 15 lbs.

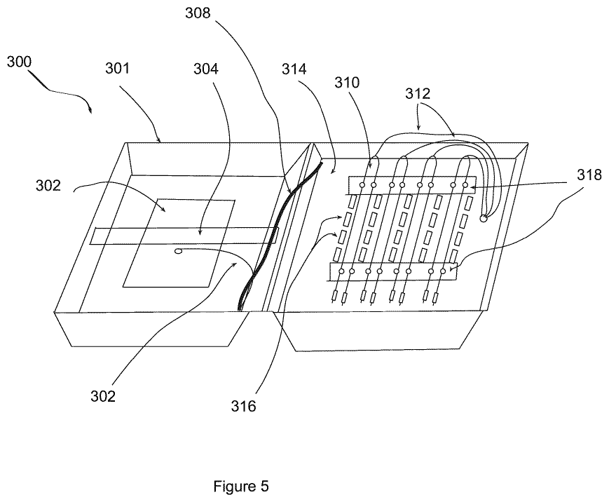

FIG. 5 is an image of an embodiment of an Ozone Generator 300. In this embodiment, within case 301, a fan (not shown) behind plate 302 is supported by support member 304. Cables 306 and 308 provide electrical power to the fan and the lamps 310, respectively. Cables 306 and 308 may be covered by an insulation and/or protective material configured to withstand the corrosive environment of the Ozone Generator 300. In some embodiments the insulation and/or protective material on cables 306 and 308 can withstand up to and including 10,000 hours of operation. Lamps 310 are shown as held in place by lamp fixture 318. Beneath the lamps 310 is shown wall 314 having openings 316.

During operation, the case 301 is closed. The fan draws ambient air into the case 301. The lamps 310 operate to generate electromagnetic energy, which induces ozone generation. Because of the air flow caused by the fan, the ozone is forced through the openings 316 and out of the case. In addition, each of a plurality of optical conductors 312 carry light safely visible from outside the case, which indicates that the lamps 310 are operating.

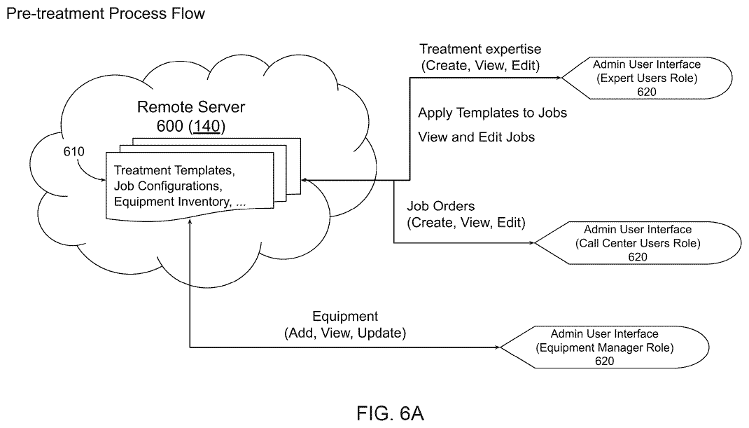

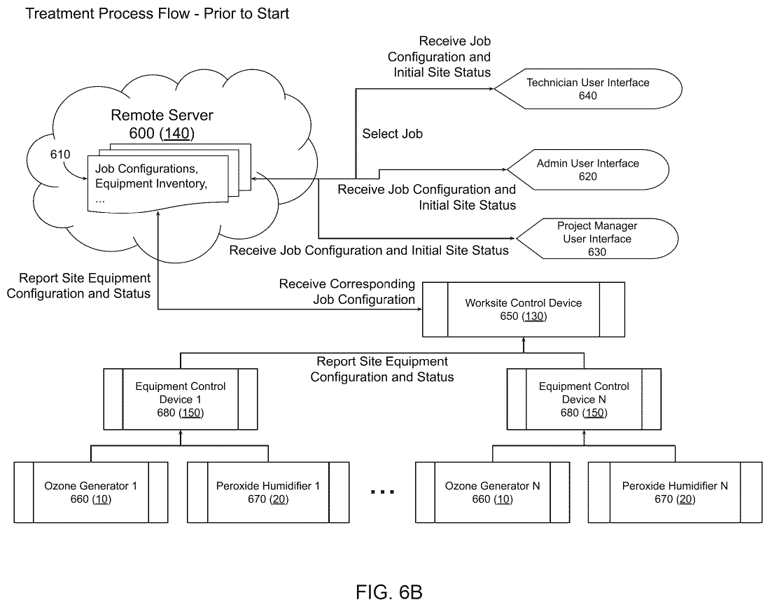

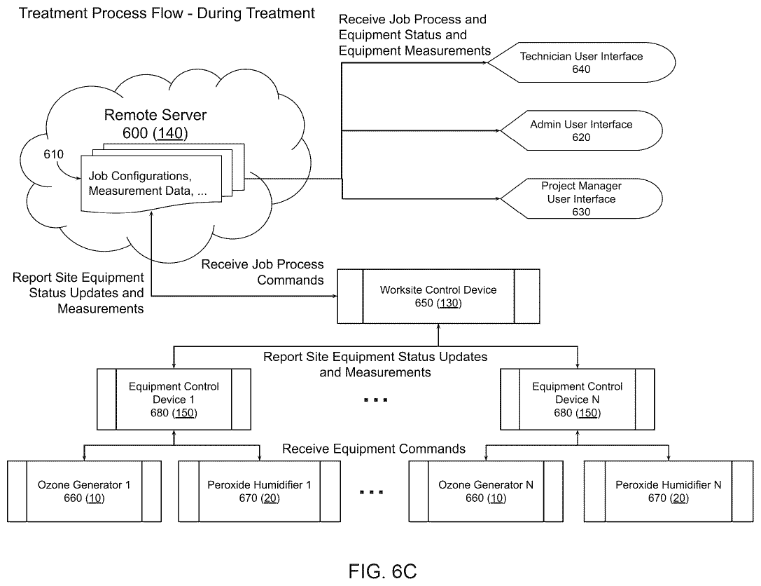

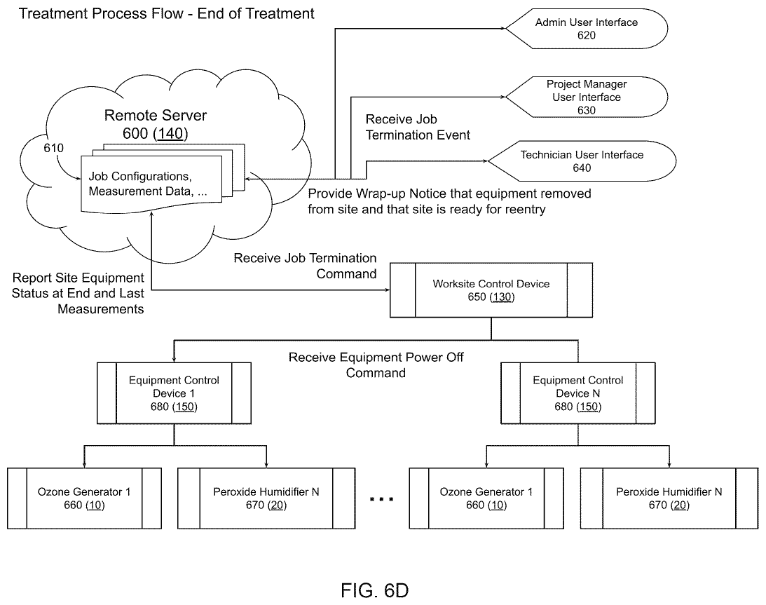

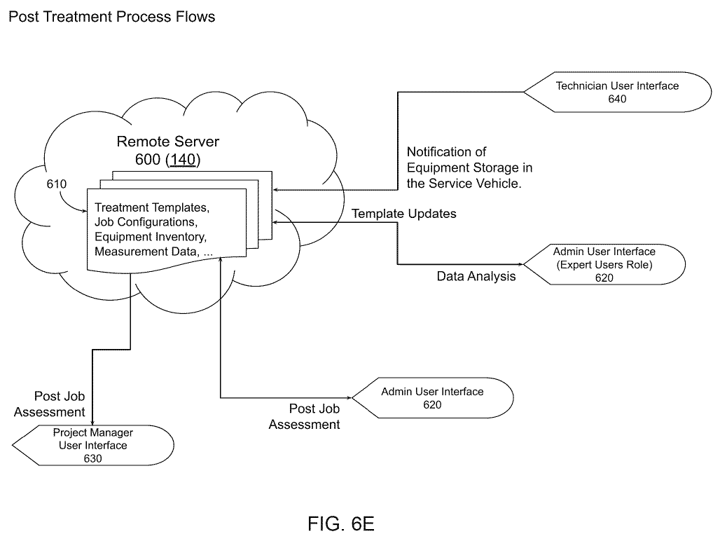

FIGS. 6A-6E are diagrams of post-treatment process flow, treatment process flow prior to start, treatment process flow during treatment, treatment process flow end of treatment and post treatment process flow.

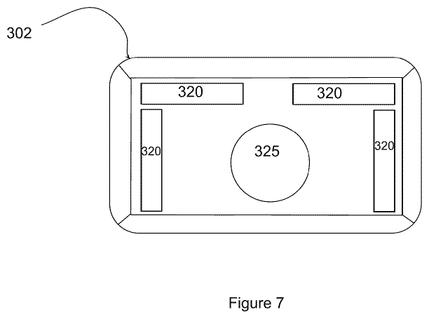

FIG. 7 is an image of case 301 of Ozone Generator 300 illustrated in FIG. 5, where the lamps 310 and the walls have not yet been installed. The case 301 has power sources 320 for the lamps and an opening 325 through which the generated ozone exits the case 301.

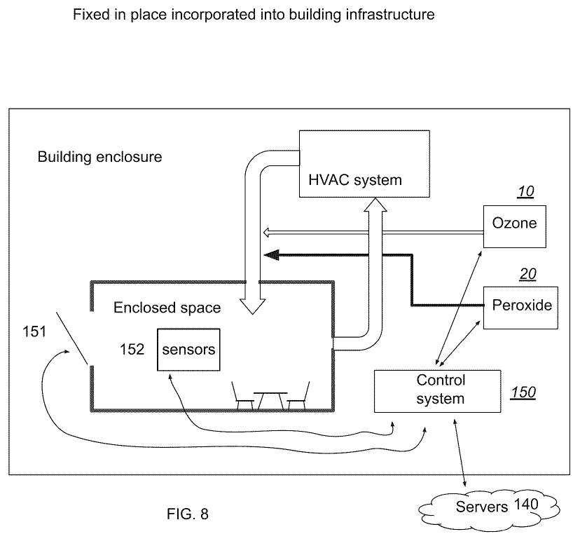

FIG. 8 depicts the fixed building installation concept where the ozone generation subsystem 10 and the hydrogen peroxide subsystem 20 are incorporated into the building and supplied via the HVAC system to an enclosed space. The control system 150 and the server 140 along with the operator interface located outside the enclosed space. The entry door 151 controls access to the enclosed space during the treatment process. Sensors inside the enclosed space relay information to the control system incorporating the same sensor combinations used in the portable concept.

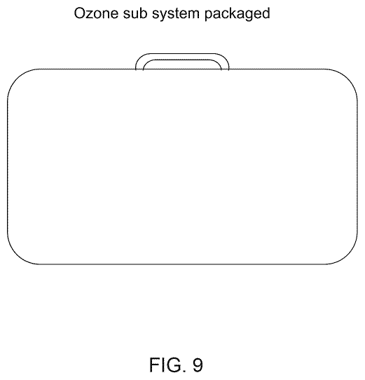

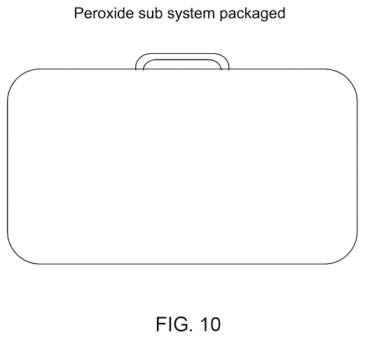

FIGS. 9 and 10 depict a case which can be used for a Peroxide Humidifier and/or an Ozone Generator case. In some systems the cases used for the Peroxide Humidifier and the Ozone Generator are substantially identical. The case may be deformable and may be formed of a material comprising at least one of a plastic, a polymer, polyethylene, and polypropylene. In some embodiments, the case has an inner and an outer layer of material with a gap therebetween. In some embodiments, the outer layer is thinner and more flexible than the inner layer.

The cases allow for axial air flow into and out of the cases. In some embodiments, the cases are sized so as to correspond to a standard racking system in a transport vehicle.

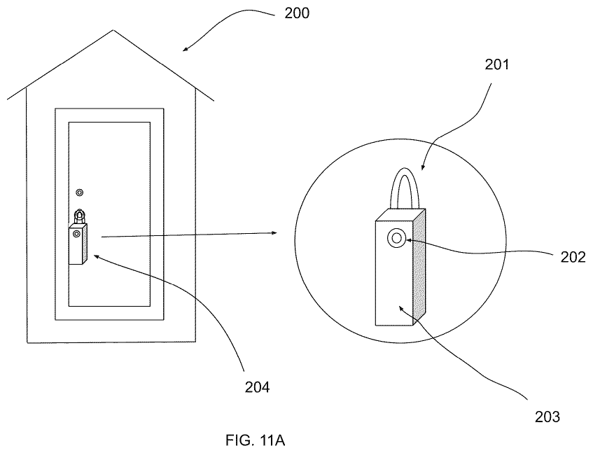

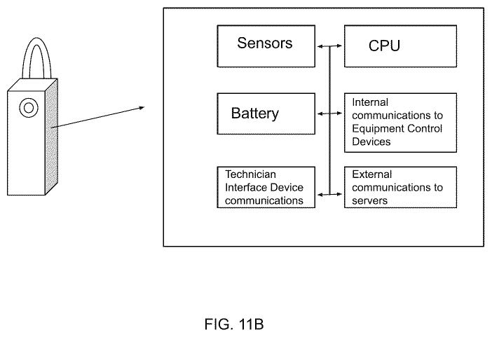

FIGS. 11A and 11B depict an embodiment of a lock box for the Worksite Control Device. As shown, this embodiment is configured to be stored on a doorknob on an enclosed space 210 and may include combination lock 202 and electronics 203 (contained within the lock box 201). System 203 contains the worksite control device 130 and consists of the items shown in FIG. 11B As discussed above with regard to the system FIG. 4, the lock box illustrated in FIGS. 11A and 11B may be in communication with one or more of a Peroxide Humidifier and an Ozone Generator. The lock box 204 may further be in communication with other local or remote equipment. The lock box may also be in communication with a network via a wired or wireless connection.

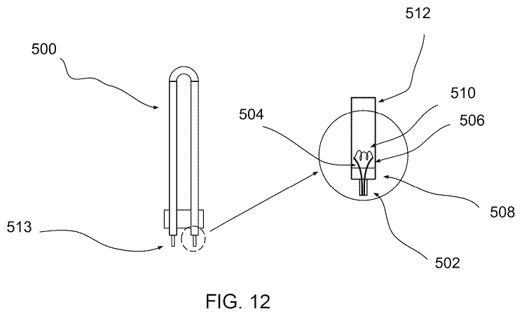

FIG. 12 is a schematic illustration of inventive aspects of a lamp which can be used, for example, with the Ozone Generator of FIG. 2. Lamp 500 includes a pin 502, wires 504, electrodes 506, filament 510, base 508, and glass 512. In some embodiments, the glass 512 comprises quartz.

The pin 502 electrically connects the lamp 500 to a power source. Wires 504 conduct current to and from the electrodes 506, which extend through the glass 512 and electrically and mechanically contact the filament 510. The base 508 provides mechanical structure and connects the pin 502 and the glass 512.

In operation, current flows through the pin 502, the wires 504, and the electrodes 506 to the filament 510. From the filament 510, the current arcs through gas within the glass 512 to another end of the lamp 500, where there is present another filament, additional electrodes and wires, and another pin 513.

In conventional lamps, there is only one electrode connecting to the filament. Accordingly, conventional lamps suffer from poor reliability. The connection between the filament and the electrode is susceptible to breakage as a result of mechanical shock. However, in certain embodiments it may be acceptable to employ conventional lamps.

Lamp 500 includes two electrodes 506, which are each connected to filament 510. Because the filament 510 is connected at both ends, it receives mechanical support at both ends, and is, therefore, much more resistant to breakage as a result of mechanical shock. In addition, if the filament 510 were to break at one end or somewhere in the middle, the lamp 500 would continue to function, because the filament or filament portions would still be connected to the electrodes 506 at the end opposite the point of breakage.

The filament 510 can be of any gauge. In some embodiments, filament 510 is medium gauge. In some embodiments, filament 510 is heavy gauge.

In some embodiments, the lamp 500 uses light weight and/or high frequency electronic ballasts to limit current.

### Abated Substances

The systems of preferred embodiments can be employed to abate any of the substances discussed herein, but are particularly preferred for abatement of pathogens, molds, allergens, and Volatile Organic Compounds (VOCs).

Pathogens that can be controlled include, but are not limited to Anthrax (Bacillus anthracis); Botulism (Clostridium botulinum toxin); Brucella species (brucellosis); Brucellosis (Brucella species); Burkholderia mallei (glanders); Burkholderia pseudomallei (melioidosis); Chlamydia psittaci (psittacosis); Cholera (Vibrio cholerae); Clostridium botulinum toxin (botulism); Clostridium perfringens (Epsilon toxin); Coxiella burnetii (Q fever); E. coli O157: H7 (Escherichia coli); emerging infectious diseases such as Nipah virus and hantavirus; Norwalk virus; Severe Acute Respiratory Syndrome (SARS); Acquired Immune Deficiency Syndrome (AIDS) virus; Human Immunodeficiency Virus (HIV); Epsilon toxin of Clostridium perfringens; Food safety threats (e.g., Salmonella species, Escherichia coli O157: H7, Shigella); Francisella tularensis (tularemia); Glanders (Burkholderia mallei); Melioidosis (Burkholderia pseudomallei); Plague (Yersinia pestis); Psittacosis (Chlamydia psittaci); Q fever ((′oxiella burnetii); Ricin toxin from Ricinus communis (castor beans); Rickettsia prowazekii (typhus fever); Salmonella species (salmonellosis); Salmonella Typhi (typhoid fever); Salmonellosis (Salmonella species); Shigella (shigellosis); Shigellosis (Shigella); Smallpox (variola major); Staphylococcal enterotoxin B; Tularemia (Francisella tularensis); Typhus fever (Rickettsia prowazekii); Variola major (smallpox); Vibrio cholerae (cholera); Viral encephalitis (alphaviruses [e.g., Venezuelan equine encephalitis, eastern equine encephalitis, western equine encephalitis]); Viral hemorrhagic fevers (filoviruses [e.g., Ebola, Marburg] and arenaviruses [e.g., Lassa, Machupo]); Water safety threats (e.g., Vibrio cholerae, Cryptosporidium parvum); bacterial responsible for necrotizing fasciitis; methicillin-resistant Staphylococcus aureus (MRSA); vancomycin-resistant Enterococcus (VRE); bacteria or mycoplasma causing pneumonia; bacteria responsible for nosocomial infections; and Yersinia pestis (plague); Covid 19 and variants.

Common household molds that can be remediated using systems of preferred embodiments include, but are not limited to Acremonium; Alternaria; Aspergillus fumigatus; Aspergillus niger; Aspergillus species Var. 1; Aspergillus species Var. 2; Aureobasidium; Bipolaris; Chaetomium; Cladosporium; Curvularia; Epicoccum; Fusarium; Geotrichum; Memnoniella; Mucor; Mycelia sterilia; Nigrospora; Paecilomyces; Penicillium species Var. 1; Penicillium species Var. 2; Pithomyces; Rhizopus; Sporothrix; Sporotrichum; Stachybotrys; Syncephalastrum; Trichoderma; and Yeast. Molds need high humidity levels and a surface on which to grow. Common areas for mold growth are garbage containers, food storage areas, upholstery, and wallpaper. Molds also commonly grow in damp areas such as basements, shower curtains, window moldings, and window air conditioners.

Indoor allergens that can be remediated by using systems of preferred embodiments include dust mite feces. Dust mite feces are the major source of allergic reactions to household dust. The mites thrive on shed human skin and are most commonly found in bedrooms, where skin cells are abundant. Preventive measures include frequently laundering bed linens in hot water and removing carpets from the room. In some cases, homeowners might have to encase the bed mattress, box springs, and pillows in vinyl covers. Other allergens of animal origin include skin scales shed from humans and animals. Commonly called dander, these are another major allergen. Dander from such animals as cats, dogs, horses, and cows can infest a home even if the animal has never been inside. Rodent urine from mice, rats, and guinea pigs is another allergen. Cockroach-derived allergens come from the insect's discarded skins. As the skins disintegrate over time, they become airborne and are inhaled.

The systems of preferred embodiments can also have utility in treating for bed bugs, fleas, ticks, mosquitoes, flies, spiders, ants, cockroaches, and other insects.

Tobacco smoke, engine exhaust, and similar allergens and odors or odor-causing agents can be abated using systems of preferred embodiments, as can volatile organic compounds from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, and the like. VOCs include organic solvents, certain paint additives, aerosol spray can propellants, fuels (such as gasoline, and kerosene), petroleum distillates, dry cleaning products, and many other industrial and consumer products ranging from office supplies to building materials. VOCs are also naturally emitted by a number of plants and trees. Some of the more common VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, and methyl ethyl ketone.

Odors and odor-causing substances that can be abated include skunk odors, urine, pet odors, and the like.

It is generally preferred to subject the substance to be abated or remediated to ozone at the preferred concentrations in the atmosphere as discussed below, generally 2 to 10 ppm, and adjust the length of treatment as necessary to ensure satisfactory kill and/or neutralization levels. Serious mold infestations are generally the most resistant substance to remediate. Treatment times of 1, 2, 3, 4, 5, 6, 12, 24, or 48 hours or more can be employed to ensure penetration of the ozone/hydroxyl mixture throughout the entire mass of a serious infestation and achieve a 100% kill and neutralization. However, should it be difficult to have the contaminated premises vacated for these long periods of time, it may be necessary to leave the treatment time at the preferred treatment time (2 to 4 hours) while at the same time increasing the level of ozone and/or hydrogen peroxide. Ozone levels of 20, 30, 40, 50, 100, 200, 400 or more ppm can be desirable along with a proportionate increase of hydrogen peroxide to such that the concentration (by weight) of hydrogen peroxide in the atmosphere is up to about 150% or more of that of ozone in the atmosphere introduced into the area to be treated, preferably from about 75% to about 150%, more preferably from about 85% to about 125%, and even more preferably from about 90% to about 100%. However, in certain embodiments it can be desirable to provide even higher concentrations of ozone.

Treatment times may vary from as short as fifteen minutes to as long as twenty-four hours and beyond, based on the allergen, pathogen, or substance being addressed, as well as its density and the surface area. A treatment time of 2 to 3 hours when using systems of preferred embodiments is generally effective in abating serious mold infestations. However, an individual mold spore is generally killed and neutralized within minutes. Protein based allergens are generally neutralized within minutes. Bacteria are generally killed after an exposure time of minutes or less. Viruses are generally killed after an exposure of less than a minute, typically after exposure times as short as several seconds. Certain molds, bacteria, allergens, and viruses can be more resistant to ozone treatments than others. For example, anthrax spores have a hard coating that is preferably “softened up” by exposure to humidity prior to ozone treatment to ensure that all spores are destroyed by a subsequent ozone treatment. See, e.g., R. G. Rice, Ozone Science and Engineering, Vol. 24, pp. 151-158 (2002). In using systems of preferred embodiments, it is generally preferred to employ treatment times of 2 to 3 hours, since such times are generally satisfactory for abating a mold infestation, and well exceed the lower limit of treatment time for substances such as protein-based allergens, bacteria, and viruses. However, when it is desired to abate a particularly virulent pathogen, such as anthrax, it can be desired to employ a treatment time of over 3 hours, for example, a treatment having a duration of 5, 6, 7, 8, 9, 10, 11, 12, 16, 20, 24, 28, 32, 36, 40, 44, 48 hours or more. It is also desirable, when abating a particularly virulent pathogen, to also increase the level of ozone and/or hydrogen peroxide. Ozone levels of 20, 30, 40, 50, 100, 200, 300, or 400 ppm or more can be desirable and the concentration by weight of hydrogen peroxide can be increased to more than 100%, preferably more than 125%, and most preferably more than 150% of the concentration by weight of ozone introduced in the area to be treated. Any suitable combination of increased time, increased relative concentration of hydrogen peroxide, and/or increased ozone concentration can be employed.

The use of systems of preferred embodiments is generally successful in abating substances that are sourced indoors, for example, a mold infestation, dander from a companion animal living in a house, mite feces, tobacco smoke, volatile organic compounds from newly installed carpeting or freshly painted walls, and the like. Substances from outside sources, such as pollen, automobile or diesel exhaust, and the like, can also be treated, but recurring treatments can be necessary as such substances reenter the interior space from outside.

Areas and Materials that can be Treated

Any interior or contained space is amenable to treatment using systems of the preferred embodiments. For example, single family homes, apartment buildings, office buildings, schools, hospitals, post offices, locker rooms, restaurants, ships, trains, buses, airplanes, trucks, recreational vehicles, mobile homes, manufactured houses, cargo containers, food processing plants, clean rooms, and the like are particularly well-suited to treatment. The systems are particularly well suited for use in newly constructed homes, buildings, vehicles, and the like, which generally contain substantial quantities of VOC sources, such as newly installed carpeting and flooring, fresh paint, adhesives, and the like. Larger enclosed spaces, such as warehouses, barns, chicken houses, and other buildings housing farm animals, grain elevators, factories, hangars, subway systems, air terminals, and the like, can also be treated provided that the preferred levels of ozone, hydrogen peroxide, temperature, and humidity can be attained. In certain embodiments, rather than seal and treat the entire volume of enclosed space, the space can be partitioned so as to maintain the preferred levels of ozone, hydrogen peroxide, temperature, and humidity in areas adjacent to those to be treated. For example, plastic sheeting can be draped over a floor or wall to be treated so as to contain the ozone/hydrogen peroxide and humidity and maintain the temperature adjacent to the treated area.

The systems of preferred embodiments can also be employed to treat materials. Materials that can be treated include any materials that can tolerate exposure to the ozone, hydrogen peroxide, humidity, and temperature conditions of preferred embodiments without suffering damage. For example, clothing, bedding and linens, rugs, mail, packages, documents, furniture, food items, agricultural products such as seeds, grains, cut flowers, produce, fruits vegetables, and live plants, containers and packaging materials, and the like. A suitable chamber can be constructed that can be sealed to maintain conditions of ozone and hydrogen peroxide concentration, humidity and temperature at preferred levels, and the material placed inside that chamber and subjected to treatment. In an automated process, materials can be moved through an airlock and into the chamber for treatment for a suitable time period, and then moved out of the chamber through another airlock. Such automated processes can be particularly well suited for the decontamination of large volumes of mail for pathogens such as anthrax, or the decontamination of animal carcasses or meat products (beef, pork, poultry, seafood, and the like) for pathogens such as salmonella or E. coli. If the treatment chamber is of sufficient size, vehicles such as passenger cars or trucks hauling various cargo, rail cars, and the like can be treated therein.

In another embodiment, FIG. 8, it can be preferred to subject a room or space to periodic decontamination. Some embodiments include a fixed in place arrangement of subsystems so the system is dedicated to repeated applications on a regular basis, such as a surgical suite in a hospital, a treatment or waiting room in a clinic, a kitchen or a restaurant, a bar or nightclub, a theater, a bingo hall, a meat processing area of a grocery store, or the like. In such embodiments, it is generally preferred to permanently install equipment in a location adjacent to the space to be treated. Such equipment can include a control unit, security devices 151, an oxygen concentrator 10, 110, an Ozone Generator, a Peroxide Humidifier/hydrogen peroxide source 20, 120, a heater and/or air conditioner HVAC and a ventilation unit. Prior to treatment at a convenient time (for example, after work hours), the space is scanned to ensure that no personnel are present in the room. Motion detectors, heat detectors, video cameras and the like can be suitable for such purposes 152. This information can be communicated to a live operator by communication methods described herein 150. Once the space has been confirmed to contain no personnel, a lock down procedure is instituted to prevent anyone from entering the space during the treatment and to maintain conditions within the space. Treatment is then conducted according to preferred embodiments using computer communication protocols to initiate the treatment process. After ozone levels have been reduced to acceptable levels, the space is then unlocked. If it is necessary to reduce ozone levels to acceptable levels in rapid fashion, ozone destruct units can be employed. A computer can be employed to control the lockdown and treatment process, as well as the treatment schedule. Another method is to quench the remaining ozone in the enclosed space by applying additional hydrogen peroxide to consume remaining ozone to acceptable levels.

### Assessment of Conditions

In preferred embodiments, an assessment of conditions in the premises is typically conducted to determine, e.g., pathogen, allergen, or gas levels, followed by treatment with ozone/hydrogen peroxide. An assessment is typically conducted to discover if a premises (e.g., house, office building, boat, and the like) has a pathogen, allergen, or other problem that can be eliminated by using the abatement systems of preferred embodiments. If it is determined that the problem can be effectively eliminated, abatement can be conducted. It is also recommended that the underlying problem responsible for the mold infestation be identified and eliminated, so as to prevent future infestations. As part of the assessment, mold tests, e.g., tests for specific types of molds can be conducted. Other testing can include tests for VOCs, tests for allergens, tests for pathogens, and the like. Ambient conditions, including temperature and relative humidity, the size of the area to be treated (square footage, volume), can also be measured. During the assessment process, it is preferred to wear appropriate protective gear, e.g., respirators, ear plugs, gloves, foot coverings, clothing coverings, goggles, and the like. For example, when dealing with an extensive infestation of particularly toxic mold, it is generally preferred to wear full hazardous material protective gear. In situations wherein the premises are subject to odors that are unpleasant but not otherwise harmful, a respirator, or no protection at all can be sufficient.

While assessments are typically conducted, in certain embodiments an assessment may not be necessary. For example, when an obvious mold infestation is present, when elimination of odors or allergens is the major impetus behind the treatment, or when the premises are treated on a periodic basis for chronic conditions such as asthma triggered by dust mites, the treatment can be initiated without performing any prior assessment.

### Preparations Before Treatment

Before commencing an abatement process or other process according to preferred embodiments, it is preferred to determine the area or volume of the premises to be treated such that the target dosage, the quantity, and type of treatment materials and equipment that is sufficient to complete the abatement process can be determined.

It is preferred to meet with the owner (or occupier) of the building before commencing the abatement process. During the meeting, the process can be explained, and the owner can assist in preparations for the abatement process. For example, all individuals, unless provided with appropriate protective gear, are instructed to leave the premises for the duration of the abatement process. Any animals, such as pets, are removed from the premises, and it is preferred to remove plants. It is not necessary to remove fish. Problem areas can be identified for treatment, along with areas that may not be amenable to treatment using the systems of preferred embodiments, or areas wherein a contamination can reoccur if the source of the contamination is not eliminated. Areas not amenable, due to either the location and/or extent of the infestation, can necessitate more extensive treatment or remediation steps, such as those employed to remove and/or dispose of toxic waste, e.g., procedures similar to those used in asbestos abatement.

### Pretreatment

In certain embodiments, it can be desirable to pre-treat the area prior to subjecting the area to ozone treatment. A preferred pretreatment involves subjecting the area to be treated with humidity. Certain pathogens, such as mold spores and anthrax, are resistant to conventional ozone treatment. The anthrax bacterium, for example, possesses a hard shell that resists penetration by ozone. By subjecting anthrax to humidity prior to ozone treatment, the ozone, and/or hydroxyl is better able to penetrate the microorganism and destroy it. Likewise, mold spores are resistant to penetration by ozone, but can be made more amenable to treatment by first subjecting them to humidity. Treatment of VOCs is also facilitated by humidity. Ozone reacts with the atmospheric water and hydrogen peroxide to produce reactive hydroxyl groups, which then react with certain VOCs to yield less harmful or harmless substances.

When the material to be treated includes molds, a pretreatment consisting of exposure to a relative humidity of 70% to 95% is typically employed. Typical pretreatment exposure times of 10 minutes, 15 minutes, 30 minutes 45 minutes, 1 hour, 1.5 hours, 2 hours, 2.5 hours, 3 hours, 4 hours, or more can be employed, depending upon the substances to be abated and the nature of the space or material to be abated. In residential mold remediation, pretreatment times of from 30 minutes to 2 hours are generally preferred. In decontaminating a material infested with anthrax, pretreatment times of from 12 hours to 24 hours are generally preferred.

### Abatement of Living and Working Spaces

If the area to be treated has an air duct system (e.g., heating or heating/air conditioning system), it is preferred to position one or more Ozone Generators and Peroxide Humidifiers adjacent to the return air inlet. Typically, for treating volumes of 25,000 cu. ft. or less, it is preferred that at least 10 g/hr of ozone is drawn into each air inlet. However, in certain embodiments satisfactory results can be obtained at a lower level of ozone generation, for example, at about 0.1, 0.5, 1, 2, 3, 4, 5, 6, 7, 8, or 9 or more g/hr. Likewise, in certain embodiments a higher level of ozone generation can be preferred, for example, about 10, 11, 12, 13, 14, 15, 20, 25, 30, 40, 50, 60, 70, 80, 90, or 100 or more g/hr. Along with the higher level of ozone generation is a proportionately higher level of hydrogen peroxide introduction, generally at a concentration by weight that is 75% to 150% of concentration by weight of ozone provided. Commercially available Ozone Generators are available in a variety of sizes. Size is generally reported in terms of ozone output in grams/hour. It is generally preferred to locate mid-sized (e.g., 6. 10 or 15 gm/hr) Ozone Generators in larger areas, e.g., living room, kitchen/family room, open stairways, open office spaces, and the like. Smaller Ozone Generators (e.g., 1-5 gm/hr) are preferably situated in small or closed-in areas, or areas that are unlikely to get circulating air, for example, basements, storage areas, and the like. Generally, dosage calculations show that Ozone Generators that are capable of generating 60 g/hr of ozone are sufficient to treat a 2,000 sq. ft. house with forced air ducting. More ozone can be preferred for a house without forced air ducting. Typically, dosage calculations show that one gram of ozone per hour is preferably delivered for every 250 cu. ft. of area to be treated. More or less ozone can be desirable, depending on humidity, temperature, and specific condition being treated. Therefore, for each 1 gm/hr of delivered ozone, an area of 33 sq. ft. with 8 ft ceilings can be decontaminated. Determination of the amount of ozone required is made according to the target dosage found in the dosage tables relating time of treatment to the ozone concentration, delivered hydrogen peroxide, humidity and temperature to the problem be treated such as mold, allergens, pathogens, or VOCs.

After the Ozone Generators are situated and turned on, the forced air system is then turned on to circulate the air. It is generally preferred that no heating or cooling of the air is conducted, however, in certain embodiments it can be preferred to heat or cool the air so as to obtain optimal abatement results.

Humidity is a significant factor in the kill rate of allergens and pathogens. In general, the higher the humidity, the faster the ozone kills the pathogen or destroys the allergen. While not wishing to be bound to any particular theory or mechanism, it is believed that the ozone reacts with the water vapor forming hydroxyl radicals thereby increasing the effectiveness of the process. Generally, it is preferred that the relative humidity in the premises to be treated be at least 30% or more, preferably the relative humidity is at least 40%, 45%, 50%, 55%, or 60% or more, more preferably the relative humidity is from 65% to about 70%, 75%, 80%, 85%, or 90% and most preferably the relative humidity is from about 70% to about 90% or 95%. Relative humidities greater than 95%, especially relative humidities of 100%, are generally not preferred due to the risk of condensation, which can lead to bleaching of sensitive materials. However, in certain embodiments relative humidities greater than 95% can be acceptable if sensitive materials are not a concern. In order to ensure that there is adequate formation of hydroxyl radicals, specifically at low humidity levels, hydrogen peroxide is preferably supplied to the area being treated at a concentration by weight that is up to about 150% or more (preferably about 75% to about 150%) of the concentration by weight of ozone being delivered. For example, if the area is being treated with about 60 g/hr of ozone, then from about 45 to 90 g/hr of hydrogen peroxide is also delivered. At low humidities, and for the abatement of particularly virulent pathogens, it is generally preferred to employ higher relative concentrations of hydrogen peroxide, preferably greater than 100%, 125%, or 150% or higher.

In coastal areas, ambient humidity can provide optimal results. In these situations, the ultrasonic Peroxide Humidifiers are used only to deliver a minimal amount of hydrogen peroxide. However, in desert areas or under low humidity conditions (e.g., winter in northern areas of the United States), it can be preferred to increase the humidity via one or more ultrasonic Peroxide Humidifiers so as to achieve optimal results. Once treatment is completed, it may be desired to employ one or more dehumidifiers to rapidly restore the ambient humidity to the treated premises, if the premises are humidity-controlled. In certain instances, it may not be feasible to humidify the area to be treated. For example, the area can contain humidity-sensitive materials (e.g., antiques, rare books, old documents, fragile textiles, or wallpaper and the like). In those instances, treatment can be conducted under ambient humidity conditions with the addition of hydrogen peroxide, but the duration of the ozone treatment can be extended to ensure satisfactory results. However, under these conditions it can be desirable to increase the ratio of delivered hydrogen peroxide to ozone from 150% to 200%, 250%, 300%, 350%, 400%, or even 1000% or more of the concentration by weight of ozone present. Any suitable schedule of introducing ozone, moisture, and/or hydrogen peroxide can be employed, e.g., constant introduction of one or more of ozone, moisture, and hydrogen peroxide, intermittent introduction of one or more of ozone, hydrogen peroxide, and humidity, varying concentrations, varying temperatures, and the like.

Temperature levels also correlate with the effectiveness of the treatment method in killing mold. Generally, as temperature increases, the effectiveness of the treatment increases. However, the amount of ozone required to achieve and maintain the target dosage also increases as the ozone more readily reverts back to oxygen at higher temperatures (i.e., ozone exhibits a shorter half-life at higher temperatures). Generally, it is preferred to conduct the treatment at a temperature typically considered a “room temperature,” namely about 17.7° C. (64° F.), 18.3° C. (65° F.), 18.8° C. (66° F.), 19.4° C. (67° F.), 20° C. (68° F.), 20.5° C. (69° F.), or 21.1° C. (70° F.) up to about 21.6° C. (71° F.), 22.2° C. (72° F.), 22.7° C. (73° F.), 23.3° C. (74° F.), 23.8° C. (75° F.), 24.4° C. (76° F.), 25° C. (77° F.), 25.5° C. (78° F.), 26.1° C. (79° F.), 26.6° C. (80° F.), 27.2° C. (81° F.), 27.7° C. (82° F.), 28.3° C. (83° F.), 28.8° C. (84° F.), or 29.4° C. (85° F.). In most residential and commercial settings, the ambient temperature falls within this range. However, if the premises to be treated are not equipped with heating or air conditioning, it can be preferred to adjust the interior temperature prior to initiating treatment, or to control the temperature at a pre-selected level during treatment. When the ambient temperature is high and the structure to be treated is not equipped with air conditioning, an air conditioning unit can be provided as part of the equipment system and used to cool the temperature, e.g., down to below 29.4° C. (85° F.). Cooling the interior to below 17.7° C. (64° F.) generally results in only an incremental reduction in the rate of ozone decomposition. Thus, it is generally not preferred to cool the interior below this temperature. If the ambient temperature is substantially below 17.7° C. (64° F.), it is generally preferred to heat the interior. In certain conditions, the temperature in the structure to be treated can be controlled to a preselected temperature, for example, a cold storage locker or a room containing equipment or machinery that must be operated at an elevated or reduced temperature. Under such conditions, the treatment is preferably conducted at ambient temperature and the ozone level and/or humidity is adjusted to achieve optimum results. In certain embodiments, however, it can be desirable to treat an area at temperatures outside of those typically considered ambient temperatures. For example, a refrigerated unit maintained at a temperature above 0° C. (32° F.) can be satisfactorily treated by adjusting the humidity, hydrogen peroxide, and ozone levels. Generally, ozone levels are increased at low temperatures. However, lower ozone levels of 2 to 10 ppm can be employed in conjunction with a longer treatment time.

Ozone levels of 2 to 10 ppm are generally preferred for treating mold and other contaminants. However, in certain embodiments it can be preferred to employ ozone levels of 1.9, 1.8, 1.7, 1.6, 1.5, 1.4, 1.3, 1.2, 1.1, 1.0, 0.9, 0.8, 0.7, 0.6, 0.5, 0.4, 0.3, 0.2 ppm or less. In other embodiments, ozone levels of 11, 12, 13, 14, 15, 16, 17, 18, 19, 20, 25, 30, 35, 40, 45, 50 ppm or more can be preferred. At optimal hydrogen peroxide, humidity, and temperature levels, a longer treatment time is preferably employed at reduced ozone levels and a shorter treatment time is preferably employed at higher levels so as to ensure a satisfactory kill and/or neutralization level.

In terms of the optimal combination of ozone level, hydrogen peroxide, temperature, and humidity, it is generally preferred to conduct a treatment at a temperature of about 21.1° C. (70° F.), a relative humidity of about 85%, a hydrogen peroxide addition of 107% by weight of ozone, and an ozone level of about 10 ppm. Under these conditions, the length of time required to achieve a 100% kill for mold spores is minimized. For in a typical residential setting, a 100% kill can be achieved in about 3 hours or less. When the temperature ranges from about 15.5° C. (60° F.) to about 26.6° C. (80° F.), i.e., interior temperatures typically observed for residential and commercial buildings, it is preferred that the relative humidity be in the range of about 70% to about 85%, and the ozone levels be in the range of about 2 ppm to about 10 ppm (with a corresponding hydrogen peroxide concentration by weight of 75% to 150% of that of the ozone concentration by weight). Under these conditions, the optimal time to achieve a 100% kill is typically about 1 to about 3 hours.

For structures situated in high humidity environments, e.g., the coastline, the Midwest during summer, and the like, wherein the relative humidity ranges from about 85% to about 95% and ambient temperatures range from about 21.1° C. (70° F.) to about 32.2° C. (90° F.), lower ozone levels can be employed. Under these conditions, the optimal time to achieve a 100% kill is typically about 1 to about 3 hours.

For structures situated in low humidity environments, e.g., desert communities, and the like, wherein the relative humidity ranges from about 5% to about 20% and ambient temperatures range from about 23.8° C. (75° F.) to about 37.7° C. (100° F.), it is preferred that the relative humidity is raised via the use of an ultrasonic Peroxide Humidifier or other suitable method to from about 70% to about 85% before beginning treatment and that the ozone levels are in the range of about 2 ppm to about 10 ppm. Under these conditions, the optimal time to achieve a 100% kill and/or neutralization is typically about 1 to about 3 hours. If it is not feasible to raise the humidity levels to the 70% to 85% range, then a higher level of hydrogen peroxide can be introduced to ensure the production of sufficient hydroxyl radicals to complete the decontamination process. Hydrogen peroxide levels of 125% to 150% of the weight of introduced ozone can generally be used to ensure decontamination. However, under the most extreme conditions it may be desirable to increase the ratio of delivered hydrogen peroxide to ozone from 150% to 200%, 250%, 300%, 350%, 400%, or even 1000% or more.

In commercial and residential settings, and the like it is preferred to open all closet and cupboard doors, and to move items stored in cupboards and closets to facilitate air circulation. Doors, windows, fireplace dampers, and other air egresses are preferably closed.

In residential settings, if dust mites are problematic, it is preferred that all linens be taken off beds and washed in 60° C. (140° F.) water. Mattresses are typically removed from the box spring and leaned up against the box spring so as to facilitate air circulation around the mattress. If feasible, blankets, pillows, and bedspreads are preferably placed in a manner that allows satisfactory air circulation. Exhaust fans, e.g., in the kitchen, bathroom, and the like, are turned on, which helps ensure that ozonated air reaches the outlet ducting of these areas. If such fans have a variable speed, they are preferably operated at their lowest possible level to reduce the amount of ozone that will be evacuated while at the same time ensuring that the vent system is decontaminated.

Before the Ozone Generators are activated, ultrasonic Peroxide Humidifiers may be employed to achieve required relative humidity levels and to deliver the hydrogen peroxide in prescribed amounts according to the target dosage. Once target humidity levels are achieved, the area can be evacuated of any nonessential personnel. For those individuals remaining in the premises, a respirator (and goggles if the respirator is not a full-face respirator) is preferably in place before turning on the Ozone Generators or hydrogen peroxide dispensers. The Ozone Generators and Peroxide Humidifiers supplying hydrogen peroxide are typically turned on remotely starting with the most remote areas of the premises and finishing with the heating and air conditioning inlet or inlets last. After the Ozone Generators have operated for a short time period, typically about ten minutes, it is preferred to test and record ozone, temperature, and humidity levels. For non-automated Ozone Generators this will require reentering the premises and taking the necessary readings. Computer controlled equipment activation is the preferred method of reducing time and effort involved and greatly improves the safety aspect of conducting them in a startup process. Once the ozone and humidity levels have reached target dosage levels, typically at least about 2 to 10 ppm ozone and 50-90% RH, effective treatment has begun, and the premises should be left closed for the duration of the treatment. Although 2 to 10 ppm ozone is generally preferred, in other embodiments a higher or lower ozone level can be desirable, e.g., less than 0.1, 0.5, 1, 2 ppm ozone up to about 15, 20, 30, 40, 50, 60, 70, 80, 90, 100, 500, 1000, 2000, 3000, 4000, 5000 ppm ozone or more. For these higher levels of ozone, proportionately higher levels of hydrogen peroxide are also generally preferred, again based on the 75% to 150% hydrogen peroxide to ozone by weight. If the contamination is particularly extensive or the mold to be abated particularly toxic, higher ozone levels can be preferred. To prevent injury, all doors and other possible entrances to the building are preferably locked and caution signs placed at the entrances.

Humidity, temperature, and ozone concentration readings are typically recorded in each room during the course of the treatment procedure. These recordings can be obtained automatically, e.g., by stand-alone recorders in the rooms or by the Ozone Generator equipped with measuring devices. Ideally, the computer systems described within would be allowed to log a majority of the reading normally done manually by an operator. Alternatively, the recordings can be obtained manually at pre-selected intervals.

Treatment is typically continued for up to 48 hours depending on the target dosage which relates temperature, humidity, ozone concentration, and condition being treated to treatment time. After treatment is completed, the Ozone Generators and Peroxide Humidifiers are turned off and ozonated air can be evacuated from the premises if immediate occupancy is desired. Alternatively, the ozone can be left to degrade back to oxygen if immediate occupancy is not required. If immediate occupancy is desired fans are typically placed in one or more doorways and/or windows to blow ozonated air out of the premises. High volume fans (9400 cfm.) are generally preferred for residential applications; however, fans capable of moving more air or less air than 9400 cfm. can also be suitable for use. The evacuation fans are typically operated for 30 minutes, and then ozone levels are tested, especially in areas where circulation from the fans is lowest, such as bedrooms, basement, and the like. Depending upon the circulation efficiency, longer or shorter operation times can be preferred. Deploying fans to evacuate vapors is not the preferred situation as it requires operators to be exposed to oxidizers which requires the use of personal protective gear with an air supply and face protection. Electronically deactivating ozone systems and activating more peroxide vapor is a much more advantageous process that can be initiated by an operator interface device and the communications protocols described in this document.

As an alternative to the use of evacuation fans, the premises can remain closed and the ozone can dissipate and/or decompose to safe levels without taking active steps to remove ozonated air from the premises. In an enclosed space with poor air circulation, ozone levels will typically return to ambient levels after about 6 hours. However, it is generally preferred to take active measures to evacuate ozonated air such that the delay in reoccupying the premises is minimized.

Ozone levels are periodically tested until a pre-selected ozone level at which it is safe to reoccupy the building is achieved. A self-contained breathing apparatus or respirator is preferably employed for testing until a level of 0.1 ppm ozone, 0.05 ppm ozone, or less is recorded, or until levels of ozone similar to outside ambient levels are achieved, if ambient levels are higher than 0.1 or 0.5 ppm. An ozone level of 0.05 ppm has been determined by the FDA to be a safe level for continuous exposure. An ozone level of 0.1 has been determined by OSHA to be safe for exposure times of up to 8 hours. Once the level drops below 0.05 ppm or outside ambient levels, the treated area is typically safe for reoccupation by people, animals, and plants and treatment is complete. If the level is not below 0.05 ppm in all areas, circulation by fans is continued until this level is reached. While 0.05 ppm is the preferred level deemed safe for reoccupation, in certain embodiments it can be preferred that a lower level be attained, e.g., a level characteristic of ambient ozone levels prior to treatment. Alternatively, a higher level of ozone can be acceptable in certain embodiments.

Ozone levels can be tested using Both UV sensors and diffusion-based sensors. Prefer a portable model commercially available instrumentation, such as that manufactured by Eco Sensors of Santa Fe, NM. When testing for current ozone levels, the instrument is used in accordance with the manufacturer's instructions. These typically include allowing adequate warm up time (generally at least 5 minutes), not blocking the air flow into the instrument while testing, making all measurements in still air as moving air can affect the readings, keeping the instrument away from the body as body odors can bias the reading, not using the instrument to take measurements directly from the outlet of the Ozone Generators which can result in incorrect readings and/or damage to the instrument.

After the premises have been deemed safe for reoccupation, doors and windows can be unlocked, caution signs can be removed, and all equipment, including fans, Ozone Generators, and Peroxide Humidifiers can be removed from the premises.

It is noted that in the case of allergens and pathogens, treatment does not remove the allergen or pathogen from the treated area. In the case of a pathogen, such as mold spores, the organism is killed. If the pathogen has an ability to produce an allergic reaction, this ability is also neutralized. In the case of an allergen such as animal dander or dust mite feces, the protein causing the allergic reaction is neutralized by the ozone treatment such that it is unable to cause an allergic reaction. A subsequent cleaning step to remove the dead organism or deactivated allergen can be desirable in certain embodiments but is not necessary.

### Pretreatment and Post-Treatment for Ozone Control

After an ozone treatment is administered and ozone returns to ambient levels, a strong ozone odor can still be noticeable. If there is such an odor, it is generally associated with closets and bedrooms. While not wishing to be bound to any particular theory, it is believed that fabrics or other materials containing natural or synthetic fibers having an electrostatic charge can attract and hold ozone, slowly releasing it back into the surrounding air at safe but noticeable levels. While a large portion of the population considers ozone to have a pleasant odor, some individuals consider the odor unpleasant. Other individuals, typically those suffering from asthma, can find ozone to be an irritant. With time, any ozone present will eventually dissipate without the need for specific measures; however, a method for reducing or eliminating lingering ozone or the odor associated with ozone is preferably employed in certain embodiments.

Any suitable method can be employed for destroying or removing lingering ozone.

Removal of ozone can also be accelerated by subjecting the interior spaces to elevated temperatures, for example, by a radiant heater or hot air blower.

In other embodiments, it can be preferred to employ ions. Depending upon the nature of the materials in the treated space, it can be desirable to employ only positive or negative ions, or to employ positive ions for a time followed by negative ions, or vice versa. Any suitable equipment for generating ions can be employed. It is generally preferred to employ an ion generator capable of producing 1·10¹² ions per second (negative or positive). Such an ion generator is generally suitable for use in rooms or spaces having an area of approximately 500 sq. ft. Alternatively, bipolar ionization can be employed. Bipolar ionization uses an alternating current to produce both positive and negative ions. Bipolar ionization utilizes a process involving association and dissociation to generate a highly reactive mixture of ionized gas consisting of atoms, molecules, and free radicals capable of creating chemical changes. There are several types of devices that can be used for this process. For HVAC applications, a non-thermal type of surface discharge reactor is preferably used. Bipolar ionization was first used commercially in 1972 in the food and meat industry in Western Europe to improve shelf life of perishable foods with limited or no mechanical refrigeration.

When the bipolar ion generator is connected to an oscilloscope, a sinusoidal waveform is observed. On one side of the waveform, the bipolar generator produces positively charged ionized gas molecules and on the other side of the waveform, the bipolar generator produces negatively charged ionized gas molecules. This is a pulsed AC system, which alternately produces negative and positively ionized gas molecules. In operation, a pulsed ion field is created in the vicinity of the bipolar generator. As air passes through the ion field, electrons in the valence shells of stable molecules receive excitation energy. As the air stream moves out of the ion field and through the air-handling unit, the electron vibrational energy permits valence electrons to overcome nuclear attraction and escape. Chemical bonds are broken in gas molecules, ionic compounds dissociate to positive and negative ions, and covalent compounds dissociate to free radicals. In the absence of a polar field, the highly unstable ions and free radicals combine to form more stable compounds.

To determine what type or types of ions are preferred for treating a space, the materials contained within the space can be classified on the basis of their place in a triboelectric series. Below is a very short triboelectric series that provides an indication of the ordering of some common materials. A material that charges positive will be the one that is closer to the positive end of the series and the material closer to the negative end will charge negatively. Accordingly, to reduce the charge on the material, ions of opposite polarity can be applied. It is the work function of the material that determines its position in the series. In general, materials with higher work function tend to appropriate electrons from materials with lower work functions. Triboelectric series (from positive to negative): positive (+)>asbestos>glass>nylon>wool>lead>silk>aluminum>paper>cotton>steel>hard rubber>nickel & copper>brass & silver>synthetic rubber>Orlon>saran>polyethylene>Teflon>silicone rubber>negative (−). While such triboelectric series can be helpful in determining the preferred ion treatment, other factors can affect the preferred treatment. For example, real materials are seldom very pure and often have surface finishes and/or contamination that strongly influence triboelectrification. The spacing between materials on a triboseries does not allow one to predict with any confidence the magnitude of the charge separated. Many factors besides the difference in the electronic surface energy, including surface finish, electrical conductivity, and mechanical properties, can also strongly influence results.

In addition to controlling ozone odor, bipolar ionization methods can yield additional benefits, including microbiological control, control of other odors and gas phase chemicals, static control, and filtration enhancement. After dissipating the ionization energy, air with a balanced electrical charge remains. In the absence of any electrical charge, submicroscopic particulates are not attracted to foreign surfaces and remain airborne and naturally buoyant. Air currents established by an efficient air distribution system displace the particulates and carry them back to the filters in the air-handling unit. Particulates that pass through the filters remain buoyant on subsequent circulation cycles and are returned to the filters for another attempt at removal. With every pass through the filters, the probability increases for removal.

It is generally preferred to employ an ion treatment during the ozone treatment. However, an ion treatment can also be conducted before or after the ozone treatment, or at any suitable time.

### Remediation of Norwalk Virus

Interior spaces, such as in cruise ships, infected with Norwalk virus can be remediated using systems of preferred embodiments. The procedure generally preferred is as follows: Ships are generally constructed such that sections of the ship can be isolated from the remainder of the ship. These sections have independent heating, ventilating, and air conditioning systems (HVAC) as well as sealable doors providing total isolation. Humidification and ozonation equipment can be brought aboard the ship and placed within one or more of the isolatable sections. The ultrasonic Peroxide Humidifiers and Ozone Generators can be placed near or within the air inlets of the HVAC system and all systems placed in operation. This circulates the ozone, hydrogen peroxide, and humidity throughout the isolated section of the ship at target dosages determined to be lethal to the Norwalk and other viruses and bacteria. Once the target dosage has been achieved then the ozone can be left to decompose back to oxygen or it can be evacuated by opening the outside makeup air system to allow 100% makeup air, thereby evacuating the ozone, and allowing rapid reoccupation of the treated section.

### Remediation of Anthrax

Systems of preferred embodiments are suitable for use in decontaminating indoor areas or material surfaces contaminated with anthrax. Anthrax is an enveloped virus requiring a change from dormant state to vegetative state in order to effectively remediate the virus. The procedure generally preferred is similar to that noted for Norwalk virus, with the exception that the area to be treated is pre-treated with humidity for a period of 12 to 24 hours at levels of humidity greater than 70%. Ultrasonic Peroxide Humidifiers are placed within the area to be treated. They are turned on and allowed to operate until such time as a level of at least 90% relative humidity is achieved. Once this level has been achieved, the pretreatment period has begun. Once the pretreatment period has been concluded, the actual treatment can be completed based on target dosages for anthrax. Target dosages for anthrax require that the amount of hydrogen peroxide relative to ozone introduced via the ultrasonic Peroxide Humidifiers to the treatment area is higher than in other decontamination situations, preferably at least from about 125 to 150%, but in certain embodiments even higher. With regard to safety issues, significantly more stringent safety procedures are required when treating an area contaminated with anthrax or other particularly virulent pathogens. Scientifically accepted hazardous material safety procedures are followed strictly by all personnel involved in the decontamination procedure.

### Operation of System

In addition to the hardware components described in the above text involving FIGS. 1-11, a control layer exists to manage the hardware layer with multiple embodiments designed to enhance the safety and efficacy of the treatment process. From here on referred to as the system. The system consists of multiple hardware components and the application software which resides within those components. The method of controlling the system is to use predefined treatment templates (“templates”) that are stored as a collection in a database 610 (FIG. [6A]) and generated from a dialog with a treatment expert. These templates consist of a sequence of events which occur during the treatment process.

The events have a title, description, a start time relative to the previous event (or zero for the initial event), a duration, which component initiates the event, an indication of whether the event is recurring, and a list of components which are notified when the event occurs.

In addition to the templates, a collection of authorized users of the system is also maintained in the database. Each user has authentication credentials, name, and contact information, and an assigned role. Each role has a list of authorized uses of the system.

The system database 610 maintains a collection of sites where treatments have occurred or are scheduled to be treated. The site description includes location by address, city, state, and country along with postal code. In addition, the geological coordinates are stored as well for use in conjunction with global positioning systems. The site is further defined by its size in area and an indication of volume, along with features which may affect which treatment template should be initially applied before customization and subsequent treatment or which were present at the time of treatment. Further description indicates the date of construction and years of occupancy. The site type is also included to distinguish various residential and commercial building types. Specific areas of concern for the treatment at the site are also included in the database.

The system also maintains in database 610 a collection of all mobile and fixed equipment used for treatments. Descriptions of each equipment item include the type, unique serial number, and item history. The history includes a list of dates with corresponding equipment characteristics such as the number of hours before and after the usage on the recorded date. Some equipment types will also include other relevant measurements such as content levels before and after usage on the recorded date.

The system maintains in database 610 a collection of treatment jobs; past, present, and future. The job will include customer information and a reference to a site for the job. The job will also include dates when service was ordered, scheduled, and performed. The job will also include the events for the job which are copied from the template for the job prior to the treatment service date. These events may be customized for a specific treatment. In addition, the expected duration of the job will be recorded prior to treatment and then updated upon completion of the job. Pricing information will also be contained for subsequent billing upon job completion. Further relevant information facilitating future jobs and/or additional customer, site, and job acquisition may also be included. Job completion will generate a post job assessment that will be available for the admin user interface 620 and project manager user interface 630 (FIG. [6E]).

A scheduling component will be used to assign which equipment will be dedicated to the job before the scheduled time of the treatment. On site, the equipment may be exchanged to accommodate any equipment failures that occur. During treatments, data measurements from the equipment used will be recorded in the history for each piece of equipment. Periodically, these measurements will be used by experts to modify job templates and/or to create new templates for use in future jobs. Artificial intelligence algorithms will also be employed to perform some of these updates without intervention by the expert 620 (FIG. [6E]).

Similar machine learning both with and without expert intervention will be used to update the templates based upon criteria gleaned from the history of job execution and equipment utilization.

The interaction with the system database will be accomplished through various user interfaces. An admin component/user interface 620 (FIG. [6A]) will be used for entering in new jobs/orders in accordance with the user's role for order taking. The same admin interface will be used for users with authorization to assign a template to a job and any subsequent customizations, prior to the scheduled time for treatment. The admin component will also be used by authorized users to assign equipment prior to the scheduled data/time for the treatment/job. During treatment execution, the admin user interface will be notified of all treatment events for a selected job and provide the capability to modify the future treatment events based upon monitoring of the job events which have already occurred.

A specialized project manager user interface 630 (FIG. [6B]) will provide similar functions for a subset of the jobs created, executed, and maintained by one or more companies which license or otherwise have access to the system. Project managers will only have access to jobs for which they are authorized to create, monitor, and maintain.

A separate technician user interface 640 (FIG. [6B]) will be used to inform the system of pretreatment readiness at the site of the treatment. For mobile usage, this will include assigned equipment placement within the site along with all safety checks performed prior to the onset of the treatment as controlled by the job description defined prior to the treatment. Similarly, for fixed system usage, the technician will also verify that all safety checks have been performed. During treatment, this technician user interface will provide real-time event start and end and for recurring events, an update of the latest measurements reported by each piece of equipment. The technician will have authorization to customize some of the job description and equipment assignments. Under normal conditions, once the technician has completed the site treatment readiness functions, the technician user interface will enable remote monitoring and some control of the job according to the technician's assigned user role.

When the technician user interface 640 (FIG. [6D]) receives the event indicating treatment completion within the site, the technician begins the post-treatment process. The treatment sequence ensures that any hazardous gas will have been dissipated and that entry is safe. Post treatment, the technician opens the site, removes the equipment, and the equipment to a service vehicle for return to a storage warehouse and or the next job site. Once the equipment is returned to the service vehicle, the technician notifies the Remote Server 600 that this has been completed as shown in (FIG. [6E]). For fixed implementations, the equipment will remain in place. In this case, the technician will verify that the safety lock has been placed in the open state.

The three user interfaces are manifested in various embodiments including web interfaces using typical personal computer web browsers and native resident applications running on mobile devices. In some cases, specialized hardware with application software may be used instead. Any and all of these may be used for a specific job and or other remote component access.

The control and monitoring of the system is enabled from remote software using the job control data along with the Worksite Control Device 650 (FIG. [6B]) executing from the site-external lock box 204 (FIG. 11A). For fixed implementations the lock box, while functionally the same, will be permanently installed. The Equipment Control Devices 680 (Figures [6B], [6C], and [6D]) within the site act as the control and data interface to individual treatment components (Peroxide Humidifiers 670 and Ozone Generators 660 in FIG. [6B], [6C], and [6D]) which apply the treatment constituents such as peroxide and ozone administration. The Peroxide Humidifiers 670 are contained in 60 of FIG. 3A. The Ozone Generators 660 are contained in 61 of FIG. 3B. The Equipment Control Devices 680 communicate with the process application components (Ozone Generators and Peroxide Humidifiers) and the Worksite Control Device 650. In normal conditions, the Worksite Control Device communicates with both the interior Equipment Control Devices and the remote job control software. Commands are executed according to the job events that are directed from the remote software. Similarly, measurements and safety status from the process administration equipment are relayed to the remote software via the Worksite Control Device.

Exception handling is an integral part of the system and is distributed throughout the system components. Exceptions are anything that would deviate from the normal operation of the system. These include power outages, communications failures, and equipment malfunctions.

Prior to treatment, these exceptions can be handled by making attempts at a later time. During treatment, the remedial action is previously defined and can be employed as exceptions are detected. To address some communication outages, job control/description information is loaded onto the worksite process controller for use without relying upon the remote system software, thereby enabling continued site treatment.

Exceptions are largely detected by health monitoring functions which periodically transmit and receive a ping signal indicating that system operation is continuing as normal according to the event sequence. Absence of a ping signal will trigger the predetermined remedial action for that exception. Instead of the real-time relay of measurement and self-test data to the remote components, local storage will be used to record the data until a future time when communication is restored and the saved data can be transmitted to the remote components for recording in the job database. Power outages in most cases will be detected in the remaining powered components and signal an early termination of a job/site treatment. These events will be transmitted to the technician, admin, and project manager user interfaces so that a technician can return to the site and remove and restore the job-assigned equipment.

### Exemplary Embodiments

System 1. An air and surface treatment system, comprising: an ozone generator; a peroxide humidifier; and a control unit, wherein the ozone generator comprises communication circuitry integrated therewith, wherein the peroxide humidifier comprises communication circuitry integrated therewith, and wherein the ozone generator and the peroxide humidifier are configured to communicate with each other and/or the Equipment Control Device communication device.

System 2: System 1, configured to communicate wired or wirelessly with a worksite control device.

System 3: System 2, wherein the Worksite Control Device is configured to communicate wired or wirelessly with a central computer system or to access an internal database.

System 4: System 3, wherein the central computer system or the internal database is adapted to store process information for past, present, and/or future treatment operations.

System 5: System 3, wherein the central computer system is adapted to store one or more data selected from the group consisting of equipment sensor information, cycle start and conclusion times, scheduling, equipment information, and maintenance records in a compartmental database configured for limited access control by an outside technician performing a remediation process at a location.

System 6: System 1, wherein the peroxide humidifier is configured to diffuse hydrogen peroxide into the air.

System 7: Any of Systems 1-6, wherein the peroxide humidifier is configured to diffuse about 450 ml/hr hydrogen peroxide into the air.

System 8: Any of Systems 1-7, wherein the peroxide humidifier is configured to diffuse about 453 ml/hr hydrogen peroxide into the air.

System 9: Any of Systems 1-8, wherein the ozone generator and the peroxide humidifier are configured to communicate status information.

System 10: Any of Systems 1-9, wherein the ozone generator and the peroxide humidifier are configured to communicate through a wireless connection.

System 11: Any of Systems 1-10, wherein the ozone generator and the peroxide humidifier are configured to communicate through a wired connection.

System 12: Any of Systems 1-11, wherein at least one of the ozone generators and the peroxide humidifier comprises a sensor and is configured to communicate sensor information.

System 13: Any of Systems 1-12, wherein at least one of the ozone generators and the peroxide humidifier is electrically connected to communication circuitry external thereto.

System 14: Any of Systems 1-13, wherein at least one of the ozone generators and the peroxide humidifier weighs less than 15 lbs.

System 15: Any of Systems 1-14 further comprising at least one Worksite Control Device, and at least one Equipment Control Device configured to communicate with at least one of the ozone generators and the peroxide humidifier.

System 16: System 15, wherein the Worksite Control Device is further configured to communicate with another Worksite Control Device of a separate system.

System 17: Any of Systems 15-16, wherein the Worksite Control Device is further configured to communicate with one or more Equipment Control Device located in a building remote to the system or in a service vehicle.

System 18: Any of Systems 15-17, wherein the Worksite Control Device(s) is located within at least one locked box.

System 19: Any of Systems 15-18, wherein the Worksite Control Device communicates via a local cellular network, a wide-area network, or the internet.

System 20: Any of Systems 15-19, wherein the Worksite Control Device has a uniform resource locator (URL).

System 21: Any of Systems 15-20, wherein the Worksite Control Device has a short-range antenna, and a connection for an external extended range antenna.

System 22: Any of Systems 15-21, wherein the Worksite Control Device has one or more connections wired or wireless for at least one of a display, a keypad, a pointing device, a printer, and other equipment.

System 23: Any of Systems 15-22, wherein the Worksite Control Device is configured to operate on battery power for up to 12 hours without recharge.

System 24: Any of Systems 15-23, wherein the Worksite Control Device is portable.

System 25: Any of Systems 15-24, wherein the Worksite Control Device weighs less than 15 lbs.

System 26: Any of Systems 15-25, wherein the ozone generator comprises: a housing; a lamp within the housing, wherein the lamp is configured to emit ozone generating light; and a plurality of walls, comprising openings therein, wherein the openings form a plurality of passages for ozone generated by the light to escape from the housing, and wherein the walls prevent the light from escaping from the housing.

System 27: System 26 further comprising a sensor within the housing.

System 28: Any of Systems 26-27, wherein the device weighs less than 15 lbs.

System 29: Any of Systems 26-28 further comprising a connection for communications circuitry.

System 30: System 29, wherein the connection comprises a wireless connection.

System 31: System 29, wherein the connection comprises a wired connection.

System 32: System 29, wherein the connection comprises a USB, firewire or other commonly available interface connections.

System 33: Any of Systems 26-32 further comprising communications circuitry.

System 34: System 33, wherein the communications circuitry comprises circuitry for wireless communication.

System 35: System 33, wherein the communications circuitry comprises circuitry for wired communication.

System 36: Any of Systems 33-35 further comprising a sensor, wherein the communications circuitry is configured to communicate sensor information.

System 37: Any of Systems 33-36, wherein the communications circuitry is configured to communicate status information.

System 38: Any of Systems 26-37 further comprising a microphone connected to the housing.

System 39: Any of Systems 26-38 further comprising a speaker connected to the housing.

System 40: Any of Systems 33-39, wherein the communications circuitry is configured to communicate with hardware, a controller, or an individual.

System 41: Any of Systems 27-39, wherein the communications circuitry is configured as a node on a communications network comprising wired and wireless communication links.

System 42: Any of Systems 33-41, wherein the communications circuitry comprises a processor and memory.

System 43: System 42, wherein the processor and the memory are configured to operate a local operating system and one or more software applications.

System 44: System 42, wherein one of the software applications is configured to process data so as to generate a result and the communications circuitry is configured to communicate the result.

System 45: Any of Systems 33-44, wherein the communications circuitry is configured to operate with power from at least one of a building, an automobile power source, and a self-contained rechargeable battery.

System 46: Any of Systems 33-45, wherein the communications circuitry is uniquely identifiable.

System 47: Any of Systems 26-46, wherein the device is configured to output about 10 grams/hour of ozone.

System 48: Any of Systems 26-47, wherein the light has a first wavelength below about 200 nanometers.

System 49: Any of Systems 26-47, wherein the light has a first wavelength of about 185 nanometers.

System 50: Any of Systems 26-49, wherein the light has a second wavelength of about 254 nanometers.

System 51: System 50, wherein the openings in the walls are staggered and wherein a ratio of 185 nm energy to 254 nm energy is about 1.

System 52: Any of Systems 26-51, wherein the lamp comprises a mercury vapor lamp.

System 53: Any of Systems 26-42, wherein the lamp is in a controlled air flow environment of about 200 cfm.

System 54: Any of Systems 26-53 wherein the lamp comprises extra heavy-duty tungsten conductors.

System 55: Any of Systems 26-54, wherein the lamp comprises an 800 ma, 96-watt mercury lamp.

System 56: Any of Systems 26-55, wherein the lamp comprises a quartz u-tube about 17 inches in length.

System 57: Any of Systems 26-56, wherein the walls comprise aluminum.

System 58: Any of Systems 26-57, wherein the walls comprise a material which absorbs the light from the lamp.

System 59: Any of Systems 26-58, wherein the walls comprise a coating which absorbs the light from the lamp.

System 60: Any of Systems 26-59, wherein the lamp is mounted to the housing with a fluorosilicone isomer.

Method 61: A method of treating air, comprising: providing an ozone generator with communication circuitry; providing a peroxide humidifier with communication circuitry; sending signals from the ozone generator to the peroxide humidifier; and sending signals from the peroxide humidifier to the ozone generator.

Method 62: Any of Methods 61, wherein signals comprise status information.

Method 63: Any of Methods 61-62, wherein the secondary signal comprises status information.

Method 64: Any of Methods 61-63, further comprising diffusing hydrogen peroxide into the air with the peroxide humidifier.

Method 65: Any of Methods 61-64, further comprising diffusing about 450 ml/hr hydrogen peroxide into the air with the peroxide humidifier.

Method 66: Any of Methods 61-65, further comprising diffusing about 453 ml/hr hydrogen peroxide into the air with the peroxide humidifier.

Method 67: Any of Methods 61-66, further comprising outputting about 10 grams/hour of ozone with the ozone generator.

Method 68: Any of Methods 61-66, wherein sending the signal comprises sending the signal through a wireless channel.

Method 69: Any of Methods 61-66, wherein sending the signal through a wired channel.

Method 70: Any of Methods 61-69, wherein at least one of the ozone generators and the peroxide humidifier comprises a sensor, and at least one of the signals comprises sensor information.

Method 71: Any of Methods 61-70, further comprising sending additional signals from at least one of the peroxide humidifiers and the ozone generator to an equipment control device.

Method 72: Method 71, further comprising sending a fourth signal from a first worksite control device to a second worksite control device of a separate system.

Method 73: Any of Methods 71-72, further comprising sending a fifth signal from the worksite control device to equipment located remote to the system, optionally in a building remote to the system or in a service vehicle.

Method 74: Any of Methods 71-73, further comprising emitting ozone generating light with a lamp within the ozone generator.

Method 75: Any of Methods 71-74, further comprising sending a sixth signal from one of the peroxide humidifiers and the ozone generator to a controller or to an individual.

Method 76: Any of Methods 71-75, wherein at least one of the peroxide humidifiers and the ozone generator comprises a processor and a memory, the method further comprising the processor and the memory operating a local operating system and one or more software applications.

Method 77: Method 76, wherein one of the software applications is configured to process data so as to generate a result and one of the first and the second signals comprises the result.

Method 78: Any of Methods 71-77, further comprising operating the peroxide humidifier and the ozone generator with power from at least one of a building, an automobile power, and a self-contained rechargeable battery.

Method 79: A method of manufacturing an ozone generator device, comprising: providing a housing; mounting a lamp within the housing, wherein the lamp is configured to emit ozone generating light; and b\connecting a plurality of walls to the interior of the housing, wherein the walls comprise openings therein, and wherein the openings are configured to allow ozone generated by the light to escape from the housing and to prevent the light from escaping from the housing.

Peroxide Humidifier 80: A peroxide humidifier, comprising: a housing enclosing a first chamber and a second chamber; the first chamber adapted to hold a hydrogen peroxide solution; and the second chamber coupled to an ultrasonic mechanism configured to vaporize hydrogen peroxide in the second chamber, wherein the second chamber is in fluid communication with the first chamber and wherein the second chamber is in fluid communication with ambient atmosphere such that vaporized hydrogen peroxide can enter the atmosphere.

Peroxide Humidifier 81: Peroxide Humidifier 80, wherein the hydrogen peroxide solution is adapted to be isolated from the outside when the peroxide humidifier is upended, tilted, inverted, or placed on its side.

Peroxide Humidifier 82: A peroxide humidifier, comprising: an air inlet; an air outlet; an evaporator; a fan configured to force air from the air inlet through the evaporator and out of the peroxide humidifier through the air outlet; a chamber adapted to hold a solution; a pump; a fluid circulation path comprising the pump, the chamber, and the evaporator, wherein at least a portion of the solution exits the evaporator in the air forced through the evaporator.

Peroxide Humidifier 83: Peroxide Humidifier 82, wherein the evaporator comprises: a diffusion layer, configured to allow the air to flow therethrough and to allow the solution to flow therethrough, wherein at least a portion of the solution exits the diffusion layer in the air; and first and second air permeable layers on opposite sides of the diffusion layer.

Peroxide Humidifier 84: Peroxide Humidifier 82 or 83, wherein the evaporator further comprises first and second screens, wherein the first air permeable layer is compressed between the first screen and the diffusion layer, and the second air permeable layer is compressed between the second screen and the diffusion layer.

Ozone Generator 85: An ozone generator device, comprising: a housing, comprising: an air inlet, and an air outlet; a lamp within the housing, wherein the lamp is configured to emit ozone generating light, and wherein the lamp comprises: a glass enclosing a gas, one or more pins, configured to electrically connect to a power source, a filament within the glass, and first and second electrodes extending through the glass and electrically connected to the pin, wherein the filament is electrically and mechanically connected to the first and second electrodes; and a fan configured to force air from the air inlet out of the device through the air outlet, wherein the air exiting the device includes ozone.

Ozone Generator 86: Ozone Generator 85, wherein the filament is medium gauge.

Ozone Generator 87: Ozone Generator 85 or 86, wherein the filament comprises a first end and a second end and the filament is connected to the first and second electrodes at the first and second ends, respectively.

Method 88: A method of introducing a solution into air, the method comprising: providing a peroxide humidifier comprising an air inlet and an air outlet; pumping a solution from a storage chamber to an evaporator; collecting the solution from the evaporator in the storage chamber; and propagating air from the air inlet through the evaporator to the outlet, wherein a portion of the solution enters the air in the evaporator and exits the peroxide humidifier with the air through the air outlet.

Method 89: Method 88, further comprising forcing air to flow through a diffusion layer in the evaporator, and providing solution to flow through the diffusion layer, wherein a portion of the solution exits the diffusion layer with the air.

Method 90: Any of Methods 88-89, wherein pumping the solution comprises pumping the solution to a top of the evaporator, wherein the evaporator is vertically oriented.

Method 91: Any of Methods 88-90, wherein propagating the air comprises forcing the air with a fan.

Method 92: A method of controlling Any of Systems 26-46, utilizing a system of templates designed to reduce human exposure to the treatment process.

Method 93: Method 92, wherein the system comprises a server, a worksite control device, an equipment control device, software templates, and human interfaces, wherein components of the system cooperate to improve the safety and efficacy of the treatment process.

Method 94: Method 93, further comprising utilizing artificial intelligence software to reduce human labor involved in a treatment process.

Method 95: Method 94, wherein the system further comprises an expert software system to reduce common treatment termination errors by managing interruptions in normal lines of communication so the treatment operator can complete tasks under adverse conditions.

System 96: An air and/or surface treatment system, comprising: at least one ozone generator, each ozone generator comprising at least one sensor and communication circuitry; at least one peroxide humidifier comprising at least one sensor and communication circuitry; and at least one equipment control device comprising communication circuitry, configured to communicate with the at least one ozone generator and the at least one peroxide humidifier, the communication unit adapted for sensor monitoring, function activation, device identification, transmission of sensor data, transmission of operation status data, and receipt of operating instructions; and a worksite control device adapted for internal monitoring and decision-making capability within the air and/or surface treatment system, wherein the worksite control device is adapted to access a database storing process information, wherein the database is internal to the worksite control device or associated with a remote computer with which the worksite control device is in communication.

System 97: System 96, comprising a plurality of equipment control devices, each equipment control device paired with one ozone generator and one peroxide humidifier, wherein each equipment control device is adapted to access the other equipment control devices, thereby enabling sensor data, operation status data, and/or operating instructions to be relayed among the ozone generators and peroxide humidifiers of the system.

System 98: System 96, wherein each equipment control device is paired with one or more ozone generators and one or more peroxide humidifiers.

System 99: System 96, wherein an equipment control device is adapted to communicate with one or more worksite control devices in a same treatment area and/or a different treatment area.

System 100: System 96, wherein the worksite control device is adapted to mount on a doorknob, is battery operated, and is adapted to react to issues with operation of components of the system, power outages, and/or safety issues.

System 101: System 96, wherein the database is adapted to store one or more data selected from the group consisting of equipment sensor information, cycle start and conclusion times, scheduling, equipment information and maintenance records in a compartmental database configured for limited access control by an outside technician performing a remediation process at a location, and/or process information for at least one of past, present, and future treatment operations.

System 102: System 96, wherein the ozone generator comprises: a housing; a lamp within the housing, wherein the lamp is configured to emit ozone generating light; and a plurality of walls configured to prevent ozone generating light from directly escaping the housing, the walls comprising openings through which ozone generated by the ozone generating light to escape from the housing.

System 103: System 102, wherein the housing comprises an air inlet and an air outlet, and wherein the lamp is situated within the housing, the lamp comprising a glass enclosing a gas, one or more pins, configured to electrically connect to a power source, a filament within the glass, and first and second electrodes extending through the glass and electrically connected to the pin, wherein the filament is electrically and mechanically connected to the first and second electrodes; the air and/or surface treatment system further comprising a fan configured to force air from the air inlet out of the device through the air outlet.

System 104: System 102, wherein the lamp comprises a solid state emitter.

System 105: System 96, wherein the system further comprises an expert software system adapted to reduce common treatment termination errors.

System 106: System 96, wherein the peroxide humidifier comprises: a first chamber adapted to hold a hydrogen peroxide solution, wherein the first chamber is adapted to prevent escape of the hydrogen peroxide solution when the humidifier is upended, tilted, inverted, or placed on its side; a second chamber coupled to an ultrasonic mechanism configured to vaporize hydrogen peroxide in the second chamber, wherein the second chamber is in fluid communication with ambient atmosphere such that vaporized hydrogen peroxide can enter the ambient atmosphere from the second chamber; and a housing enclosing the first chamber and the second chamber.

System 107: System 96, wherein the peroxide humidifier further comprises: an air inlet; an air outlet; an evaporator; a fan configured to force air from the air inlet through the evaporator and out of the peroxide humidifier through the air outlet; and at least one of a fluid circulation path and a diffusion assembly; wherein the fluid circulation path comprises a pump, a chamber adapted to hold the hydrogen peroxide solution, and an evaporator, the fluid circulation path adapted such that at least a portion of the hydrogen peroxide solution exits the evaporator in air forced through the evaporator; and wherein the diffusion assembly comprises a diffusion layer, a first air permeable layer, a second air permeable layer, a first screen, and a second screen, wherein the first air permeable layer and the second air permeable layer are on opposite sides of the diffusion layer, wherein the first air permeable layer is compressed between the first screen and the diffusion layer, wherein the second air permeable layer is compressed between the second screen and the diffusion layer, wherein the diffusion assembly is configured for airflow and flow of the hydrogen peroxide solution therethrough, wherein at least a portion of the hydrogen peroxide solution exits the diffusion assembly into air.

System 108: System 96, wherein the device is configured to output about 10 grams/hour of ozone, and wherein the peroxide humidifier is configured to diffuse about 450 ml/hr hydrogen peroxide into air in the space to be treated.

System 109: System 108, wherein at least one ozone generator or at least one peroxide humidifier weighs less than 15 lbs.

System 110: System 96, wherein the peroxide generator comprises: a fill port; a first tank below the fill port and connected on a top of the first tank by a fluid channel to the fill port, the first tank comprising a vent situated on the top of the first tank and a drain situated on a bottom of the first tank; and a second tank of approximately a same volume as the first tank, the second tank positioned adjacent to the first tank, wherein a top, a bottom, a front, and a back of the second tank are approximately aligned with a top, a bottom, a front, and a back of the first tank, the second tank comprising a vent situated on a top of the second tank and a drain situated on a bottom of the second tank adjacent to a front or a back side of the second tank, wherein the front or the back side of the second tank is opposite to a back side or a front side of the first tank adjacent to which the first tank vent is situated; wherein a volume of the first tank and a volume of the second tank is a total volume; wherein the first tank is configured to receive an initial fill of hydrogen peroxide solution of less than 50% of the total volume; wherein the first tank is in fluid communication with the second tank via tubing connecting the first tank vent and the second tank vent, wherein the tubing comprises a U-bend extending to a level approximately at a midpoint of the first tank and a midpoint of the second tank, the tubing comprising a T-connection at a bottom of the U-bend wherein tubing connects the T-connection to the diffuser assembly; and wherein the first tank is in fluid communication with the second tank via tubing connecting the first tank drain and the second tank drain, wherein the tubing comprises a S-bend at a level approximately level with the bottom of the first tank and the bottom of the second tank, the tubing comprising a T-connection at a midpoint of the S-bend wherein tubing connects the T-connection to the diffuser assembly, wherein a one-way valve is placed between the diffuser assembly and the T-connection so as to prevent passage of liquid from the T-connection into the diffuser assembly while permitting passage of liquid from the diffuser assembly to the T-connection.

Method 111: A method for safely sanitizing an enclosed area, comprising: providing the system of System 96; identifying one or more predetermined treatment parameters; positioning the at least one ozone generator in an area to be treated and connecting the ozone generator to a power source; positioning the at least one peroxide humidifier in the area to be treated, connecting the peroxide humidifier to a power source, and providing the peroxide humidifier with an appropriate amount of hydrogen peroxide solution; via the worksite control device, optionally situated outside the area being treated, initiating an initial treatment protocol by sending instructions to the equipment control device to start the ozone generator and the peroxide humidifier; recording sensor data from the ozone generator and the peroxide humidifier; evaluating the sensor data by comparing it to process information stored in the database, so as to determine if the initial treatment protocol must be modified to achieve the one or more predetermined treatment parameters, wherein evaluating is performed by the worksite control device or a remote computer in communication with the worksite control device; and modifying, via the worksite control device, the initial treatment protocol to achieve the one or more predetermined treatment parameters without entering the area being treated; terminating, via the worksite control device, the treatment from outside the area being treated by sending instructions to the equipment control device to stop the ozone generator and the peroxide humidifier; determining that the area being treated is safe to enter without entering the area being treated; and reporting the determination that the area being treated is safe to enter.

Method 112: Method 111, further comprising evaluating data about the environment inside or outside of the area being treated, wherein the data is not generated by the sensor of the at least one ozone generator or the at least one peroxide humidifier.

Method 113: Method 111, further comprising: loading job information into the worksite control device; powering up and self-testing the at least one ozone generator and the at least one peroxide humidifier; establishing communications between the worksite control device and the at least one ozone generator and the at least one peroxide humidifier, wherein establishing comprises report to the worksite control device successful powering up and self-testing, retrieving job parameters by the at least one ozone generator and the at least one peroxide humidifier from the worksite control device, acknowledging receipt of job parameters by the at least one ozone generator and the at least one peroxide humidifier, and sending a ready signal from the at least one ozone generator and the at least one peroxide humidifier; and thereafter initiating a start of the at least one ozone generator and the at least one peroxide humidifier remotely.

Method 114: Method 111, further comprising: receiving, by the at least one ozone generator and the at least one peroxide humidifier, a start command whereby operation of the at least one ozone generator and the at least one peroxide humidifier is initiated; monitoring, by the at least one ozone generator and the at least one peroxide humidifier, data from the sensor, wherein monitoring comprises; recording the sensor data in the worksite control device or the remote computer, and checking the sensor data for an anomaly preventing safe operation by comparing the sensor data to process information stored in the database, and if an anomaly is found, initiating an action selected from the group consisting of stopping treatment, turning off the at least one ozone generator, turning off the at least one peroxide humidifier, adjusting a treatment protocol, sending information regarding the anomaly or action in response to an anomaly to a remote computing device, and requesting receipt of a command from a remote computing device; determining that desired treatment targets have been achieved and terminating the treatment; and sending to a remote communication device a notification that treatment has been terminated.

Method 115: Method 111, further comprising: confirming the at least one ozone generator and the at least one peroxide humidifier have ceased operation; confirming that the area treated is a safe environment; confirming that, if portable, the system of System 96 has been removed from the treatment area; and evaluating data related to the treatment by the worksite control device or remote computer, wherein evaluating includes determining future treatment modifications, equipment maintenance, or other information related to treatment protocols, and storing the data related thereto in the database.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

1. An air and/or surface treatment system, comprising:
at least one ozone generator, each ozone generator comprising at least one sensor and communication circuitry;
at least one peroxide humidifier comprising at least one sensor and communication circuitry;
at least one equipment control device comprising communication circuitry, configured to communicate with the at least one ozone generator and the at least one peroxide humidifier, the communication circuitry adapted for sensor monitoring, function activation, device identification, transmission of sensor data, transmission of operation status data, and receipt of operating instructions; and
a worksite control device adapted for internal monitoring and decision-making capability within the air and/or surface treatment system, wherein the worksite control device is adapted to access a database storing process information, wherein the database is internal to the worksite control device or associated with a remote computer with which the worksite control device is in communication.

2. The air and/or surface treatment system of claim 1, comprising a plurality of equipment control devices, each equipment control device paired with one ozone generator and one peroxide humidifier, wherein each equipment control device is adapted to access the other equipment control devices, thereby enabling sensor data, operation status data, and/or operating instructions to be relayed among the ozone generators and peroxide humidifiers of the system.

3. The air and/or surface treatment system of claim 1, wherein each equipment control device is paired with one or more ozone generators and one or more peroxide humidifiers.

4. The air and/or surface treatment system of claim 1, wherein an equipment control device is adapted to communicate with one or more worksite control devices in a same treatment area and/or a different treatment area.

5. The air and/or surface treatment system of claim 1, wherein the worksite control device is adapted to mount on a doorknob, is battery operated, and is adapted to react to issues with operation of components of the system, power outages, and/or safety issues.

6. The air and/or surface treatment system of claim 1, wherein the database is adapted to store one or more data selected from the group consisting of equipment sensor information, cycle start and conclusion times, scheduling, equipment information and maintenance records in a compartmental database configured for limited access control by an outside technician performing a remediation process at a location, and/or process information for at least one of past, present, and future treatment operations.

7. The air and/or surface treatment system of claim 1, wherein the ozone generator comprises:
a housing;
a lamp within the housing, wherein the lamp is configured to emit ozone generating light; and
a plurality of walls configured to prevent ozone generating light from directly escaping the housing, the walls comprising openings through which ozone generated by the ozone generating light to escape from the housing.

8. The air and/or surface treatment system of claim 7, wherein the housing comprises an air inlet and an air outlet, and wherein the lamp is situated within the housing, the lamp comprising a glass enclosing a gas, one or more pins, configured to electrically connect to a power source, a filament within the glass, and first and second electrodes extending through the glass and electrically connected to the pin, wherein the filament is electrically and mechanically connected to the first and second electrodes; the air and/or surface treatment system further comprising a fan configured to force air from the air inlet out of the device through the air outlet.

9. The air and/or surface treatment system of claim 7, wherein the lamp comprises a solid state emitter.

10. The air and/or surface treatment system of claim 1, wherein the system further comprises an expert software system adapted to reduce common treatment termination errors.

11. The air and/or surface treatment system of claim 1, wherein the peroxide humidifier comprises:
a first chamber adapted to hold a hydrogen peroxide solution, wherein the first chamber is adapted to prevent escape of the hydrogen peroxide solution when the humidifier is upended, tilted, inverted, or placed on its side;
a second chamber coupled to an ultrasonic mechanism configured to vaporize hydrogen peroxide in the second chamber, wherein the second chamber is in fluid communication with ambient atmosphere such that vaporized hydrogen peroxide can enter the ambient atmosphere from the second chamber; and
a housing enclosing the first chamber and the second chamber.

12. The air and/or surface treatment system of claim 1, wherein the peroxide humidifier further comprises:
an air inlet;
an air outlet;
an evaporator;
a fan configured to force air from the air inlet through the evaporator and out of the peroxide humidifier through the air outlet;
and at least one of a fluid circulation path and a diffusion assembly;
wherein the fluid circulation path comprises a pump, a chamber adapted to hold a hydrogen peroxide solution, and an evaporator, the fluid circulation path adapted such that at least a portion of the hydrogen peroxide solution exits the evaporator in air forced through the evaporator; and
wherein the diffusion assembly comprises a diffusion layer, a first air permeable layer, a second air permeable layer, a first screen, and a second screen, wherein the first air permeable layer and the second air permeable layer are on opposite sides of the diffusion layer, wherein the first air permeable layer is compressed between the first screen and the diffusion layer, wherein the second air permeable layer is compressed between the second screen and the diffusion layer, wherein the diffusion assembly is configured for airflow and flow of the hydrogen peroxide solution therethrough, wherein at least a portion of the hydrogen peroxide solution exits the diffusion assembly into air.

13. The air and/or surface treatment system of claim 1, wherein the device is configured to output about 10 grams/hour of ozone, and wherein the peroxide humidifier is configured to diffuse about 450 ml/hr hydrogen peroxide into air in the space to be treated.

14. The air and/or surface treatment system of claim 13, wherein at least one ozone generator or at least one peroxide humidifier weighs less than 15 lbs.

15. The air and/or surface treatment system of claim 1, wherein the peroxide generator comprises:
a fill port;
a first tank below the fill port and connected on a top of the first tank by a fluid channel to the fill port, the first tank comprising a vent situated on the top of the first tank and a drain situated on a bottom of the first tank; and
a second tank of approximately a same volume as the first tank, the second tank positioned adjacent to the first tank, wherein a top, a bottom, a front, and a back of the second tank are approximately aligned with a top, a bottom, a front, and a back of the first tank, the second tank comprising a vent situated on a top of the second tank and a drain situated on a bottom of the second tank adjacent to a front or a back side of the second tank, wherein the front or the back side of the second tank is opposite to a back side or a front side of the first tank adjacent to which the first tank vent is situated;
wherein a volume of the first tank and a volume of the second tank is a total volume;
wherein the first tank is configured to receive an initial fill of hydrogen peroxide solution of less than 50% of the total volume;
wherein the first tank is in fluid communication with the second tank via tubing connecting the first tank vent and the second tank vent, wherein the tubing comprises a U-bend extending to a level approximately at a midpoint of the first tank and a midpoint of the second tank, the tubing comprising a T-connection at a bottom of the U-bend wherein tubing connects the T-connection to a diffuser assembly; and
wherein the first tank is in fluid communication with the second tank via tubing connecting the first tank drain and the second tank drain, wherein the tubing comprises a S-bend at a level approximately level with the bottom of the first tank and the bottom of the second tank, the tubing comprising a T-connection at a midpoint of the S-bend wherein tubing connects the T-connection to the diffuser assembly, wherein a one-way valve is placed between the diffuser assembly and the T-connection so as to prevent passage of liquid from the T-connection into the diffuser assembly while permitting passage of liquid from the diffuser assembly to the T-connection.

16. A method for safely sanitizing an enclosed area, comprising:
providing the system of claim 1;
identifying one or more predetermined treatment parameters;
positioning the at least one ozone generator in an area to be treated and connecting the ozone generator to a power source;
positioning the at least one peroxide humidifier in the area to be treated, connecting the peroxide humidifier to a power source, and providing the peroxide humidifier with an appropriate amount of hydrogen peroxide solution;
via the worksite control device, optionally situated outside the area being treated, initiating an initial treatment protocol by sending instructions to the equipment control device to start the ozone generator and the peroxide humidifier;
recording sensor data from the ozone generator and the peroxide humidifier;
evaluating the sensor data by comparing it to process information stored in the database, so as to determine if the initial treatment protocol must be modified to achieve the one or more predetermined treatment parameters, wherein evaluating is performed by the worksite control device or a remote computer in communication with the worksite control device; and
modifying, via the worksite control device, the initial treatment protocol to achieve the one or more predetermined treatment parameters without entering the area being treated;
terminating, via the worksite control device, the treatment from outside the area being treated by sending instructions to the equipment control device to stop the ozone generator and the peroxide humidifier;
determining that the area being treated is safe to enter without entering the area being treated; and
reporting the determination that the area being treated is safe to enter.

17. The method of claim 16, further comprising evaluating data about the environment inside or outside of the area being treated, wherein the data is not generated by the sensor of the at least one ozone generator or the at least one peroxide humidifier.

18. The method of claim 16, further comprising:
loading job information into the worksite control device;
powering up and self-testing the at least one ozone generator and the at least one peroxide humidifier;
establishing communications between the worksite control device and the at least one ozone generator and the at least one peroxide humidifier, wherein establishing comprises:
reporting to the worksite control device successful powering up and self-testing,
retrieving job parameters by the at least one ozone generator and the at least one peroxide humidifier from the worksite control device,
acknowledging receipt of job parameters by the at least one ozone generator and the at least one peroxide humidifier, and
sending a ready signal from the at least one ozone generator and the at least one peroxide humidifier; and thereafter
initiating a start of the at least one ozone generator and the at least one peroxide humidifier remotely.

19. The method of claim 16, further comprising:
receiving, by the at least one ozone generator and the at least one peroxide humidifier, a start command whereby operation of the at least one ozone generator and the at least one peroxide humidifier is initiated;
monitoring, by the at least one ozone generator and the at least one peroxide humidifier, data from the sensor, wherein monitoring comprises:
recording the sensor data in the worksite control device or the remote computer, and
checking the sensor data for an anomaly preventing safe operation by comparing the sensor data to process information stored in the database, and if an anomaly is found, initiating an action selected from the group consisting of stopping treatment, turning off the at least one ozone generator, turning off the at least one peroxide humidifier, adjusting a treatment protocol, sending information regarding the anomaly or action in response to an anomaly to a remote computing device, and requesting receipt of a command from a remote computing device;
determining that desired treatment targets have been achieved and terminating the treatment; and
sending to a remote communication device a notification that treatment has been terminated.

20. The method of claim 16, further comprising:
confirming the at least one ozone generator and the at least one peroxide humidifier have ceased operation;
confirming that the area treated is a safe environment;
confirming that, if portable, the system has been removed from the treatment area; and
evaluating data related to the treatment by the worksite control device or remote computer, wherein evaluating includes determining future treatment modifications, equipment maintenance, or other information related to treatment protocols, and storing the data related thereto in the database.
